(19) United States
(12) Patent Application Publication
(10) Pub. No.: US 2023/0143628 A1
(43) Pub. Date: May 11, 2023

(54) SYSTEMS AND METHODS OF CLASSIFYING MOVEMENTS FOR VIRTUAL REALITY ACTIVITIES

(71) Applicant: PENUMBRA, INC., Alameda, CA (US)

(72) Inventors: William Ka-Pui Yee, Alameda, CA (US); Misha Tomaye Leong, Alameda, CA (US)

(21) Appl. No.: 17/520,944

(22) Filed: Nov. 8, 2021

(51) Int. Cl. G16H 20/30; G06F 16/28; G06F 16/2457; G06N 20/00; G06F 3/01

(52) U.S. Cl. CPC G16H 20/30; G06F 16/285; G06F 16/24578; G06N 20/00; G06F 3/017; G06F 3/011

(57) ABSTRACT

Systems and methods are provided for classifying movements, such as activities of daily living (ADLs) and other essential activities, using a virtual reality system. Generally, a VR system may receive input from a plurality of sensors, generate a movement signature based on the input, determine a movement classification based on the movement signature, and then provide the movement classification. The VR system may generate a movement signature based on charting position data, rotation data, and/or acceleration data, from one or more of the plurality of sensors. In some embodiments, movement classification may be performed by, e.g., using a model trained from movement data stored in a movement library. In some embodiments, VR activities may be identified and selected based on movements or micromovements desired for a patient to practice motions in ADLs. In some embodiments, potentially problematic movements may be identified based on performance of VR activities.

### BACKGROUND OF THE DISCLOSURE

The present disclosure relates generally to virtual reality (VR) systems and more particularly to capturing and classifying movements for therapeutic exercises in VR activities.

### SUMMARY OF THE DISCLOSURE

Helping patients learn or relearn to perform movements for regular everyday tasks and activities of daily living (ADLs) can be important goals for therapeutic treatment. Virtual reality systems may be used in various applications, including therapeutic activities and exercises, to assist patients with their rehabilitation and recovery from illness or injury. Sometimes, a patient may struggle with certain movements and parts of movements (e.g., micromovements) required to perform one or more ADLs. VR therapy may be used to monitor and help patients, in a safe and observable environment, to retrain their brains and muscles to perform certain tasks that may be difficult for them. For instance, VR applications can immerse a patient in a virtual world and guide the patient to perform virtual tasks to help practice movements or micromovements beneficial to ADLs. VR systems can track body motion using sensors and, e.g., using machine learning, build a movement library of ADLs and key tasks based on captured and classified movements. VR activities may involve practicing motions (and parts of motions) foundational to vital everyday tasks, and further activities may be developed to help train specific movements and micromovements found in a movement library.

In some cases, a therapist may have difficulty recognizing and/or identifying several VR activities that may be appropriate for a patient needing to exercise problematic ADL areas. A VR therapeutic movement platform can identify VR activities that feature movements and micromovements involved in challenging ADLs and tailor a therapy session to practice similar smaller movements in a game-like virtual world that would be engaging as well as entertaining. As such, the patient would not be focused on the difficulties in performing the various activities or exercises, but instead be entertained and carried away by carefully crafted virtual environment and therapeutic exercises. The VR therapeutic movement platform may identify VR activities that can direct a patient to perform ADL-critical movements (and/or parts of movements) in a manner that challenges and engages the patient physically and mentally.

ADLs are basic self-care activities necessary for independent living, e.g., at home and/or in a community. Typically, many ADLs are performed on a daily basis. For instance, some of the most common ADLs may include the activities that many people complete when they wake up in the morning and get ready to leave their home, e.g., get up out of bed, use a toilet, shower or bathe, get dressed, groomed, and eat. Generally, there are typically five basic categories, including personal hygiene, dressing, self-feeding, bathroom activities, and functional mobility. People who experience mental or physical impairments may struggle to perform some of the movements necessary to accomplish one or more of these routine daily tasks, and, thus, may not be able to live independently. For example, someone with limited shoulder mobility may have difficulty washing one's hair. A patient experiencing a movement disorder, such as Parkinson's disease, may find drinking water challenging. A woman recovering from a stroke may not be able to lift a toothbrush to brush her own teeth. Doctors and therapists, such as occupational therapists, may evaluate a patient's abilities on using tools such as the Katz Index of Independence in Activities of Daily Living. Based on measurements and diagnoses, doctors and therapists may design a course of therapy to help a patient improve the basic movements that are essential for regular as well as vital activities.

Other important activities for living independently in a community may include instrumental activities of daily living (IADLs). IADLs are considered a little more complex than ADLs and may not be necessary for fundamental functioning. The Occupational Therapy Practice Framework from the American Occupational Therapy Association (AOTA) identifies IADLs such as care of others, care of pets, child rearing, communication management, driving and community mobility, financial management, health management and maintenance, home establishment and management, meal preparation and clean up, religious and spiritual activities and expressions, safety procedure and emergency responses, and shopping. Sometimes included as IADLs are activities like, rest and sleep, education, work, play, leisure, and social participation. Not all IADLs may be absolutely vital for everyday life, but a person's inability to perform movements necessary for some IADLs may encumber his or her independence, social interactions, etc.

Understandably, there are other activities that may challenge a person outside of basic ADLs and IADLs that may help to make daily living more independent and fulfilling. Less serious injuries with shorter-term recovery may inhibit certain movements that one may be accustomed to performing, even though it may not be necessary for typical daily functioning. For instance, someone experiencing back pain may not be able to jump the same way as when they were healthy. A person recovering from a broken leg may not be able to jog. A normally high-performing athlete rehabilitating an upper body injury may not even be able to perform motions necessary for swinging, e.g., a golf club or tennis racquet. Losing abilities to perform certain movements may unfortunately necessitate losing corresponding activities in one's life.

VR activities have shown promise as engaging therapies for patients suffering from a multitude of conditions, including various physical, neurological, cognitive, and/or sensory impairments. Generally, VR systems can be used to instruct users in their movements while therapeutic VR can recreate practical exercises that may further rehabilitative goals such as physical development and neurorehabilitation. For instance, patients with physical and neurocognitive disorders may use therapy for treatment to improve, e.g., range of motion, balance, coordination, mobility, flexibility, posture, endurance, and strength. Physical therapy may also help with pain management. Some therapies, e.g., occupational therapies, may help patients with various impairments develop or recuperate physically and mentally to better perform everyday living functions, and ADLs. VR systems can encourage patients by depicting avatars performing tasks that a patient with various impairments may not be able to fully perform.

VR therapy can be used to treat various disorders, including physical disorders causing difficulty or discomfort with reach, grasp, positioning, orienting, range of motion (ROM), conditioning, coordination, control, endurance, accuracy, and others. VR therapy can be used to treat neurological disorders disrupting psycho-motor skills, visual-spatial manipulation, control of voluntary movement, motor coordination, coordination of extremities, dynamic sitting balance, eye-hand coordination, visual-perceptual skills, and others. VR therapy can be used to treat cognitive disorders causing difficulty or discomfort with cognitive functions such as executive functioning, short-term and working memory, sequencing, procedural memory, stimuli tolerance and endurance, sustained attention, attention span, cognitive-dependent IADLs, and others. In some cases, VR therapy may be used to treat sensory impairments with, e.g., sight, hearing, smell, touch, taste, and/or spatial awareness.

A VR system may use an avatar as a representation of the patient and animate the avatar in the virtual world. Using sensors in VR implementations of therapy allow for real-world data collection as the sensors can capture movements of various body parts such as hands and arms for the system to convert and animate an avatar in a virtual environment. Such an approach may approximate the real-world movements of a patient to a high degree of accuracy in virtual-world movements. Data from the many sensors may be able to produce statistical feedback for viewing and analysis by doctors and therapists. Generally, a VR system collects raw sensor data (e.g., position, orientation, linear and rotational movements, movement vectors, acceleration data, etc.) from patient movements, filters the raw data, passes the filtered data to an inverse kinematics (IK) engine, and then an avatar solver may generate a skeleton and mesh model in order to render and animate the patient's virtual avatar.

Typically, avatar animations in a virtual world may closely mimic the real-world movements, but virtual movements may be exaggerated or modified in order to aid in therapeutic activities. Visualization of patient movements through avatar animation may stimulate and promote physical and neurological repairs, recovery, and regeneration for a patient. For example, a VR therapy activity may depict an avatar feeding a squirrel some seed from a spoon held by the avatar corresponding to a patient's actual movements of grabbing and shaking a seed dispenser into a spoon held by the other hand. A VR therapy activity may ask a patient to stack virtual ingredients for a specific sandwich by requiring the patient to reach towards and grab various ingredients, such as bread, meats, cheeses, lettuce, and condiments in a step-by-step fashion. VR activities have shown promise as engaging therapies for patients suffering from a multitude of conditions, and these activities can be made to include engaging features to otherwise mentally and physically tough activities. Therapy can be stress-inducing and still can cause patient fatigue and frustration. More VR activities are being developed to address specialized impairments with tailored exercises.

With a variety of VR activities, comes a variety of exercises for therapy patients. However, not every exercise or activity is correct or properly suited for every patient. Patients may each have various physical, neurological, cognitive, and/or sensory impairments to be treated. A therapist must be cognizant of movements that a patient may have difficulty performing and adjust the therapy by, e.g., selecting more appropriate VR activities, difficulty and/or intensity levels, etc. Asking a therapist to memorize movements of each VR activity, as well as movements that may be difficult for, e.g., dozens of patients, is not feasible. There exists a need to store movements and/or lists of movements particular to each activity and relevant for each patient. There exists a need for some type of a movement library.

As disclosed herein, a movement library may be a collection of movements (e.g., including parts of movements and micromovements) and activities found in ADLs, VR activities, problematic patient motions, and other important activities. For instance, a movement library may comprise organized lists, databases, catalogs, charts, plots, graphs, images, animations, videos, and other compilations of ADLs and activities that various patients may perform on a regular basis. In some cases, motion capture sessions may be performed by a motion-capture (mocap) performer—e.g., a performer working with a doctor, therapist, VR application designer, supervised assistant, actor, or other trained professional—performing an activity, e.g., brushing teeth or drinking water from a glass, to capture proper form of the motion in a movement library. For instance, VR hardware and sensors (e.g., electromagnetic sensors, inertia sensors, accelerometers, optical sensors, and any other position, orientation, angular and rotation sensors) may be positioned on the body of the mocap performer, and she may perform the necessary steps or movements of the ADL for capture by the VR system and incorporation into a movement library. Sensors of the VR system may capture a performed movement and the VR system may translate real-world movement to movement by a VR avatar in a virtual world. Real-world motion-capture performance may be completed with or without props, inside or outside the usual setting (e.g., bathroom, kitchen). In some embodiments, a mocap performer may be able to pantomime an ADL using movements proper enough for capture and training. In some cases, portions of a movement library may be built from patient capture data.

From the data of each movement in a movement library, a movement signature may be derived for each input movement. Generally, generating a movement signature comprises generating a visual, graphical, vector map or vector chart or vector plot, mathematical representation, and/or any other similar means of data illustrations describing a movement. For instance, a movement signature may be a graph of sensor data over time. In some embodiments, generating a movement signature may include steps of plotting one or more portions of sensor data as graphs and generating a movement signature based on the graphs. Each movement signature may be stored within the movement library—and each movement may be identified based on its movement signature. In some embodiments, a movement signature may be generated based on plotting or charting one or more of position data, rotation data, and/or acceleration data, captured from one or more of the plurality of sensors, over time. For instance, a movement signature may comprise a composite of multiple sensors' position, orientation, and tri-axial accelerometer data.

Using data from a movement library, such as movement signatures, an activity classification model may be trained using machine learning techniques. For instance, inputting sensor data, VR avatar skeletal data, and/or movement signatures, a trained model can classify movement as a particular activity that was documented in the movement library previously.

As disclosed herein, a VR activity platform can classify movements, such as ADLs and essential activities, using a virtual reality system. Generally, a VR system may receive input from a plurality of sensors, generate a movement signature based on the input from the plurality of sensors, determine a movement classification based on the movement signature, and then provide the movement classification. The VR system may generate a movement signature based on the input from the plurality of sensors by, e.g., plotting time against sensor data, such as at position data, rotation data, and acceleration data, from one or more of the plurality of sensors.

In some embodiments, movement classification may be performed by, e.g., using a trained machine learning model to generate data indicative of a classification of the movement signature based on a plurality of stored movement signatures. The model may be trained to receive the movement signature as input and output at least one movement classification describing the input movement signature. In some embodiments, determining the movement classification may be performed with data analytics techniques to generate data indicative of a classification of the movement signature based on a plurality of stored movement signatures.

In some embodiments, a movement library may comprise a visual representation of each movement, such as an avatar animation performing the movement. A movement library interface, e.g., user interface, may render each movement amination when selected or may provide a demonstration activity to allow a patient to directly see and practice vital activities. Still, a therapy patient being asked to mimic or mirror specific motions may be boring and ultimately discourage focus and completion of therapy, even knowing how valuable practicing such actions may be for independent living. There further exists a need for providing VR therapy applications that help teach and develop motions for ADLs in an engaging manner.

VR therapy can be quite engaging when, e.g., using a VR activity to help develop coordination and strength to perform a real-world task. However, matching real-world tasks with motions performed in VR activities is not always easily done. For instance, practicing motions like feeding some seeds via spoon to a virtual squirrel may be helpful exercises for brushing one's teeth. Motions like grabbing a spoon handle and lifting and lowering the spoon can be analogous and beneficial to grabbing and lifting a toothbrush. Small movements like those may be considered micromovements. In some embodiments, identifying activities helpful for practicing ADL-like motions may be performed by matching micromovements between VR activities and critical real-world activities.

Generally, micromovements may be considered smaller portions of a movement required to perform the movement. For instance, a hand-washing movement by a patient (or motion capture actor) may comprise soaping his hands, lowering his hands under a faucet, and rinsing his hands in separate steps. Micromovements may be referred to as smaller movements, sub-movements, partial movements, and/or small motions. In some embodiments, signatures of micromovements may be incorporated in a movement signature. In some embodiments, micromovements, such as a lather action, a lowering action, and a rinsing action, may each have their own movement signature, e.g., within a larger movement signature, such as hand washing.

In some embodiments, micromovements may be identified within a movement signature. Extracting a micromovement from a larger movement signature may comprise identifying breaks in the movement signature. In some embodiments, breaks may be identified by, e.g., abrupt changes in position, rotation, and/or acceleration in one or more directions. For instance, a large change in height position (e.g., y-position) may indicate a lifting or lowering micromovements. A large change in horizontal position (e.g., z-position) may indicate a pulling or pushing micromovements. In some embodiments, breaks may be identified by brief or instantaneous pauses in activity. In some embodiments, breaks may be identified by changes in patterns of motion, such as when oscillating starts or stops, or when raising or lowering starts/stops. In some embodiments, a machine learning model may be trained to identify changes between micromovements within a movement. Micromovements and their respective movement signatures (and portions of full movement signatures) may be recorded in a movement library.

As disclosed herein, a VR activity platform can identify and provide one or more therapeutic activities to encourage practicing of particularly challenging movements related to ADLs and activities essential for independent living. Generally, a VR platform may receive an input associated with a first movement, determine a plurality of micromovements based on the first movement, access a plurality of activities, e.g., from a movement library, each with one or more micromovements and compare the micromovements of the first movement with the micromovements of the activities to determine an activity that is a good match. In some embodiments, the VR platform may compare the plurality of micromovements with the one or more exercise micromovements associated with each of the plurality of activities and identify a subset of the plurality of activities based on the comparison of the plurality of micromovements with the one or more exercise micromovements associated with each of the plurality of activities, then provide the subset of the plurality of activities.

As disclosed herein, a VR activity platform can classify movements, such as ADLs and essential activities, using a virtual reality system. Generally, a VR system may receive input from a plurality of sensors, generate a movement signature based on the input from the plurality of sensors, determine a movement classification based on the movement signature, and then provide the movement classification. The VR system may generate a movement signature based on the input from the plurality of sensors by, e.g., plotting time against sensor data, such as at position data, rotation data, and acceleration data, from one or more of the plurality of sensors. In some embodiments, there may be classifications, sub-classifications, as well as groups and sub-groups, and/or other hierarchies of classifying and grouping.

In some embodiments, the VR platform may then assign each of the subset of the plurality of activities a match score based on the comparison of the plurality of micromovements with the one or more exercise micromovements associated with each of the plurality of activities, rank the subset of the plurality of activities a match score based on the match score, and select an activity of the plurality of activities based on the ranking of the subset of the plurality of activities before providing the selected activity of the plurality of activities. In some embodiments, the VR platform can capture movements and micromovements of a patient, e.g., within VR activities, and compare them to one or more exemplary movement signatures to identify potentially problematic ADLs and activities.

In some embodiments, the first movement may be a movement classification determined by a trained machine learning model. The VR platform may, for instance, receive input from a plurality of sensors for a movement, generate a first movement signature based on the input from the plurality of sensors for the movement and then pass the movement signature to a trained model. The trained model may generate data indicative of a classification of the first movement signature based on training from a plurality of stored movement signatures and output the movement classification. A movement signature, in some embodiments, may be determined based on sensor input by, e.g., charting time against position data, rotational data, and/or acceleration data. In some embodiments, micromovements may have signatures that may be, e.g., found in movement signatures when compared.

With a VR platform identifying and suggesting therapy activity to aid movements and micromovements in ADLs, a therapist may be able to better focus on the patient. A VR platform may also allow a patient to independently practice portions of a guided VR activity regimen outside of a therapist's office, e.g., at home under the supervision of a family member and/or a remote supervisor.

### BRIEF DESCRIPTION OF THE DRAWINGS

The above and other objects and advantages of the disclosure will be apparent upon consideration of the following detailed description, taken in conjunction with the accompanying drawings, in which like reference characters refer to like parts throughout, and in which:

FIG. 1A is an illustrative depiction of a captured movement and a movement signature for a VR therapy platform, in accordance with some embodiments of the disclosure;

FIG. 1B is an illustrative depiction of a captured movement and a movement signature for a VR therapy platform, in accordance with some embodiments of the disclosure;

FIG. 1C is an illustrative depiction of a captured movement and a movement signature for a VR therapy platform, in accordance with some embodiments of the disclosure;

FIG. 1D is an illustrative depiction of a user interface for a VR therapy platform, in accordance with some embodiments of the disclosure;

FIG. 2A depicts an illustrative data structure for a movement library, in accordance with some embodiments of the disclosure;

FIG. 2B depicts illustrative data structures for movement data, in accordance with some embodiments of the disclosure;

FIG. 3A depicts an illustrative flowchart of a process for classifying a movement, in accordance with some embodiments of the disclosure;

FIG. 3B depicts an illustrative flowchart of a process for classifying a movement signature, in accordance with some embodiments of the disclosure;

FIG. 3C depicts an illustrative flowchart of a process for classifying a movement signature, in accordance with some embodiments of the disclosure;

FIG. 4A depicts an illustrative flowchart of a process for adding a movement to a movement library, in accordance with some embodiments of the disclosure;

FIG. 4B depicts an illustrative flowchart of a process for extracting micromovements from a movement, in accordance with some embodiments of the disclosure;

FIG. 5 depicts an illustrative flow diagram of a process for training a movement classifier model, in accordance with some embodiments of the disclosure;

FIG. 6 depicts an illustrative flowchart of a process for selecting a VR therapy activity appropriate for practicing a movement, in accordance with some embodiments of the disclosure;

FIG. 7A is a diagram of an illustrative system, in accordance with some embodiments of the disclosure;

FIG. 7B is a diagram of an illustrative system, in accordance with some embodiments of the disclosure;

FIG. 7C is a diagram of an illustrative system, in accordance with some embodiments of the disclosure;

FIG. 7D is a diagram of an illustrative system, in accordance with some embodiments of the disclosure;

FIG. 8A is a diagram of an illustrative system, in accordance with some embodiments of the disclosure;

FIG. 8B is a diagram of an illustrative system, in accordance with some embodiments of the disclosure;

FIG. 8C is a diagram of an illustrative system, in accordance with some embodiments of the disclosure;

FIG. 9 is a diagram of an illustrative system, accordance with some embodiments of the disclosure; and

FIG. 10 is a diagram of an illustrative system, in accordance with some embodiments of the disclosure.

### DETAILED DESCRIPTION

Various systems and methods disclosed herein are described in the context of a therapeutic system for helping patients with physical, neurological, cognitive, and/or sensory impairments, but this application is only illustrative. Such a VR system may be suitable for wellness and athletic pursuits or guided activities and the like, for example, coaching athletics, training dancers or musicians, teaching students, and other activities. Such systems and methods disclosed herein may apply to various VR applications. Moreover, embodiments of the present disclosure may be suitable for augmented reality, mixed reality, and assisted reality systems.

In context of the VR system, the word “therapy” may be considered equivalent to physical therapy, cognitive therapy, neurological therapy, sensory therapy, behavioral therapy, meditational therapy, occupational therapy, preventative therapy, assessment for therapies, and/or any other methods to help manage an impairment or condition, as well as a combination of one or more therapeutic programs. Such a VR system may be suitable with, for example, therapy, coaching, training, teaching, entertainment and other activities. Such systems and methods disclosed herein may apply to various VR applications.

In context of the VR systems, the word “patient” may be considered equivalent to a subject, user, participant, student, etc. and the term “therapist” may be considered equivalent to doctor, physical therapist, clinician, coach, teacher, supervisor, or any non-participating operator of the system. A therapist may configure and/or monitor via a clinician tablet, which may be considered equivalent to a personal computer, laptop, mobile device, gaming system, or display. Some disclosed embodiments include a digital hardware and software medical device that uses VR for health care, focusing on physical and neurological rehabilitation. The VR device may typically be used in a clinical environment under the supervision of a medical professional trained in rehabilitation therapy.

In some embodiments, the VR device may be configured for personal use at home, e.g., with remote monitoring. A therapist or supervisor, if needed, may monitor the experience in the same room or remotely. In some cases, a therapist may be physically remote or in the same room as the patient. For instance, some embodiments may need only a remote therapist. Some embodiments may require a remote therapist with someone, e.g., a nurse or family member, assisting the patient to place or mount the sensors and headset and/or observe for safety. Generally, the systems are portable and may be readily stored and carried by, e.g., a therapist visiting a patient.

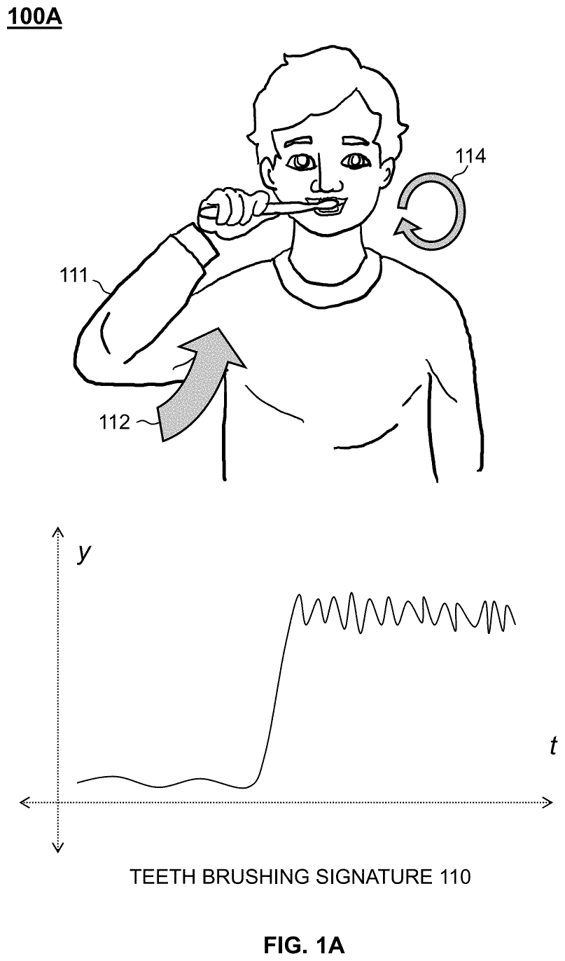

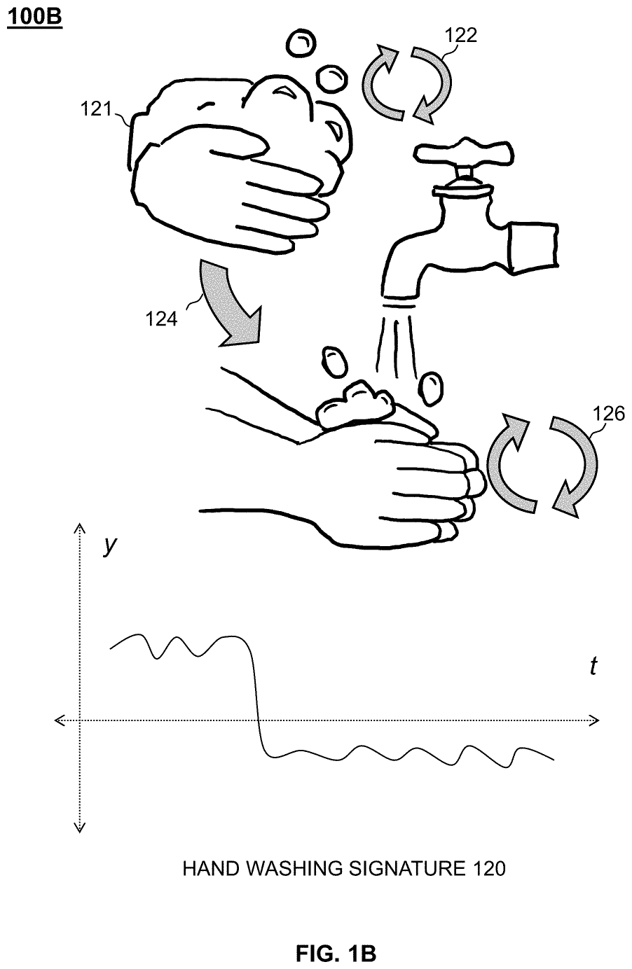

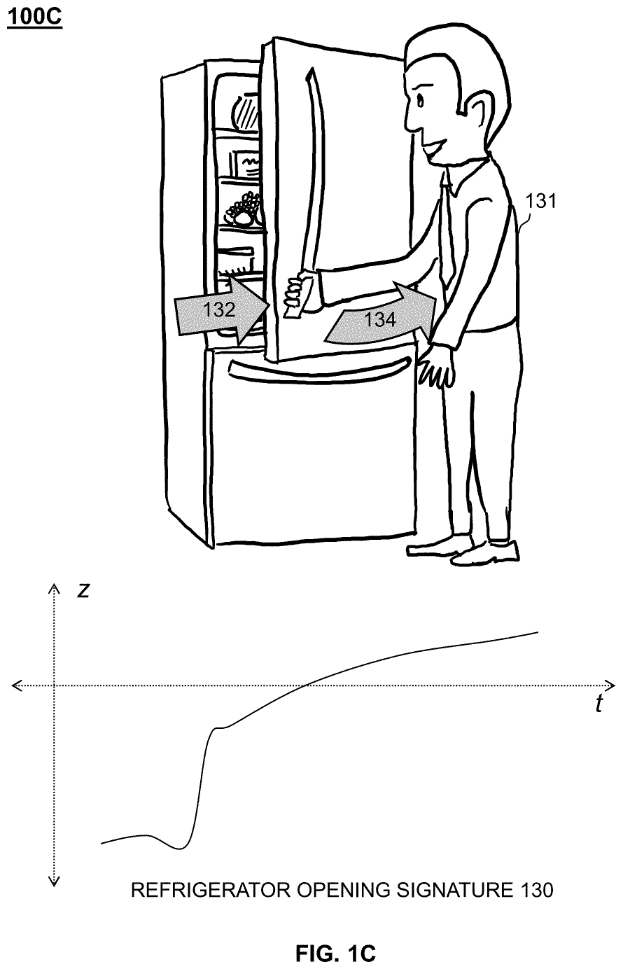

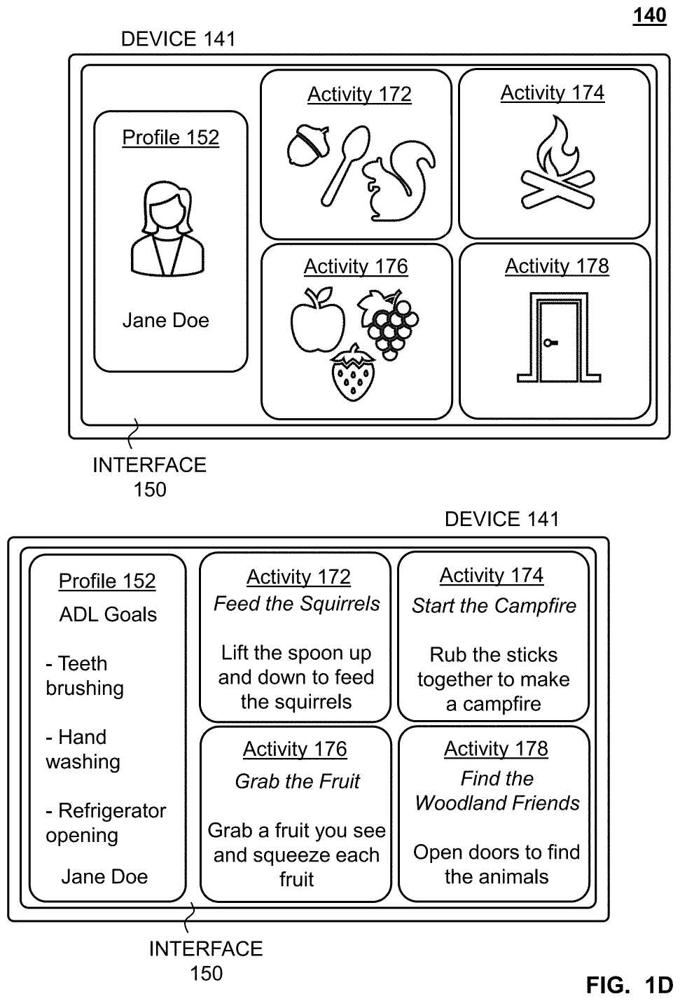

FIG. 1A is an illustrative depiction of a captured movement and a movement signature for a VR therapy platform, in accordance with some embodiments of the disclosure. Scenario 100A of FIG. 1A depicts, e.g., a VR avatar brushing his teeth. Like many other hygiene activities, teeth brushing may be considered an important activity for independent living, as well as an ADL. Depicted in scenario 100A, avatar 111 performs at least two smaller movements (e.g., micromovements) including raising action 112 and brushing action 114. The brushing action may include a number of micromovements, such as rotation of the hand, deflection of the wrist, and rotation of the forearm.

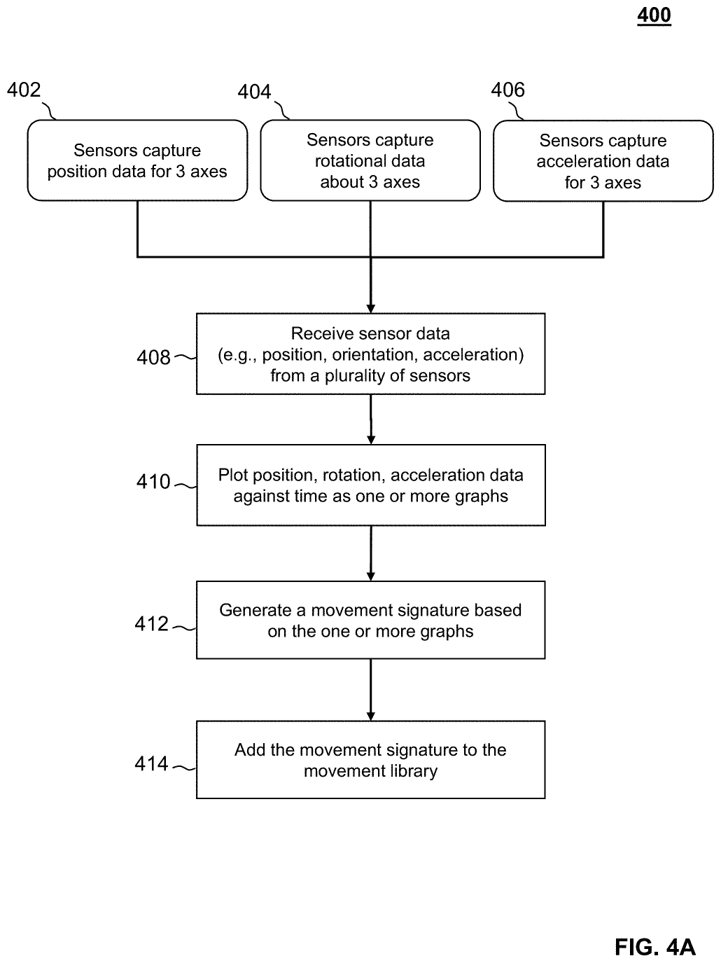

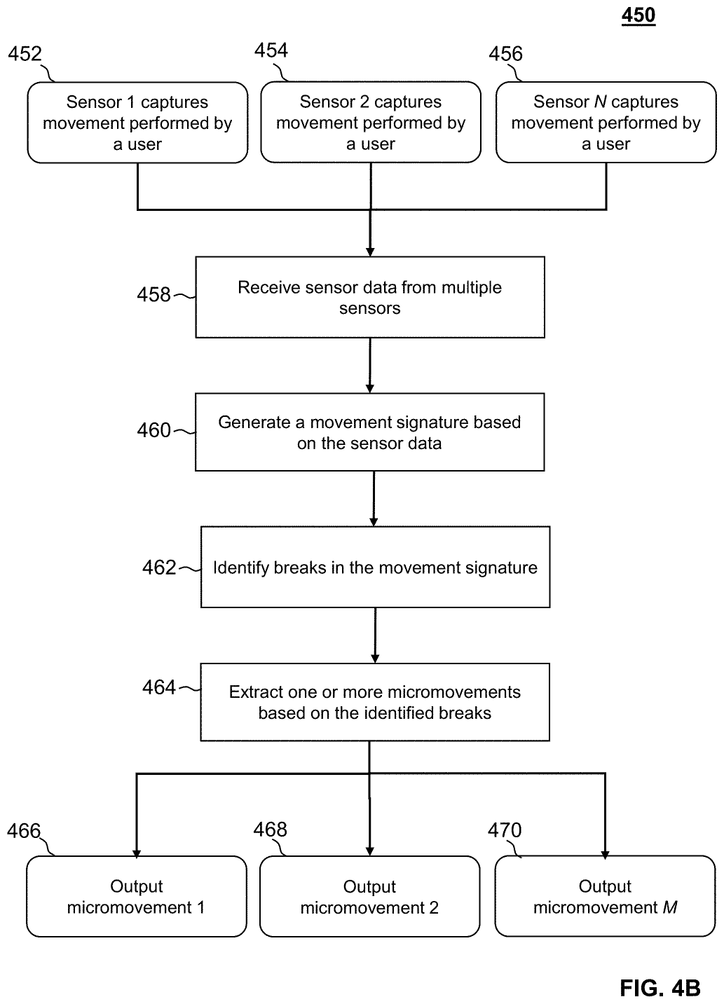

Movements such as brushing teeth may be incorporated into the VR system, e.g., as part of a movement library. In some embodiments, a movement library may include data describing proper motions for various ADLs and activities. In some embodiments, each movement of a movement library may have a movement signature. A movement library may comprise a user interface that provides an avatar animation of an ADL, e.g., scenario 100A. In some embodiments, scenario 100A may be a portion of a VR application or activity used to help train patients. Some embodiments may add a movement to a movement library through a process such as the steps depicted in FIG. 4A.

Scenario 100A of FIG. 1A may be considered a depiction of movements required to properly complete an ADL, e.g., teeth brushing, as captured by sensors positioned on a user of a VR system. Such a capture session may be performed by a motion-capture performer (a “mocap performer” working with, e.g., a doctor, therapist, VR application designer, supervised assistant, actor, or other trained professional) performing the activity, e.g., brushing teeth, to capture proper form of the motion in the VR system. For instance, VR hardware and sensors may be positioned on the body of the mocap performer, and she may perform the necessary steps of the ADL for capture by the VR system. Sensors of the VR system may capture a performed movement and the VR system may translate real-world movement to movement by a VR avatar in a virtual world. Real-world performance may be completed with or without props, inside or outside the usual setting (e.g., bathroom). In some embodiments, a mocap performer may be able to pantomime an ADL using movements proper enough for capture and training.

Scenario 100A may be displayed to a patient view via the head-mounted display, e.g., “Patient View.” Scenario 100A may also be considered a user interface of the same VR application as depicted to a spectator, such as a therapist. For instance, a spectator, such as a therapist, may view scenario 100A and see a reproduction or mirror of a patient's view in the HMD, e.g., “Spectator View.” In some embodiments, scenario 100A may be displayed as another view via a VR application interface, e.g., exploring a movement library.

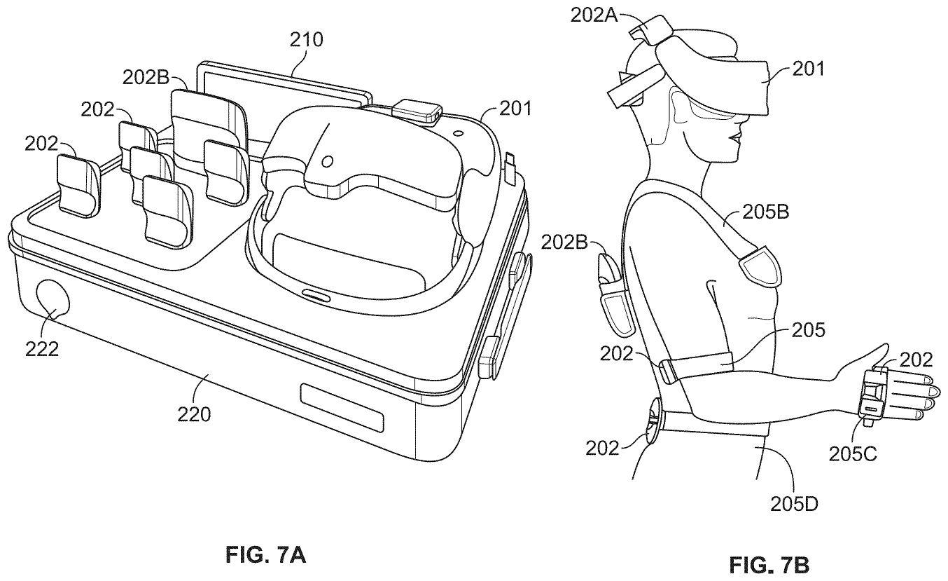

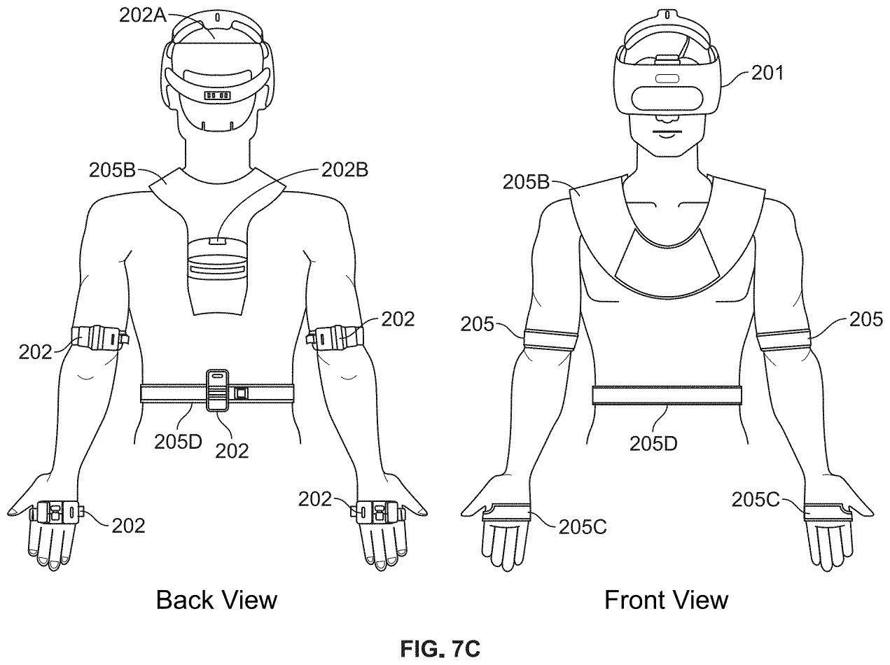

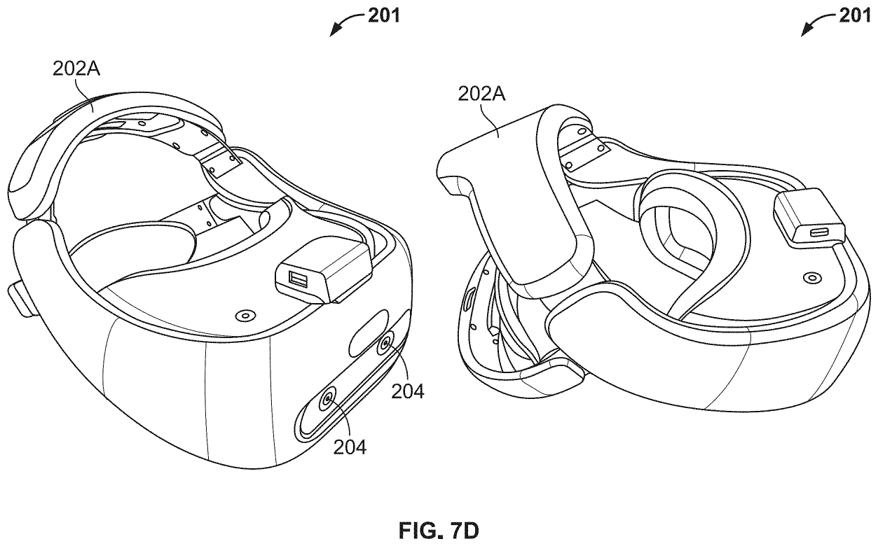

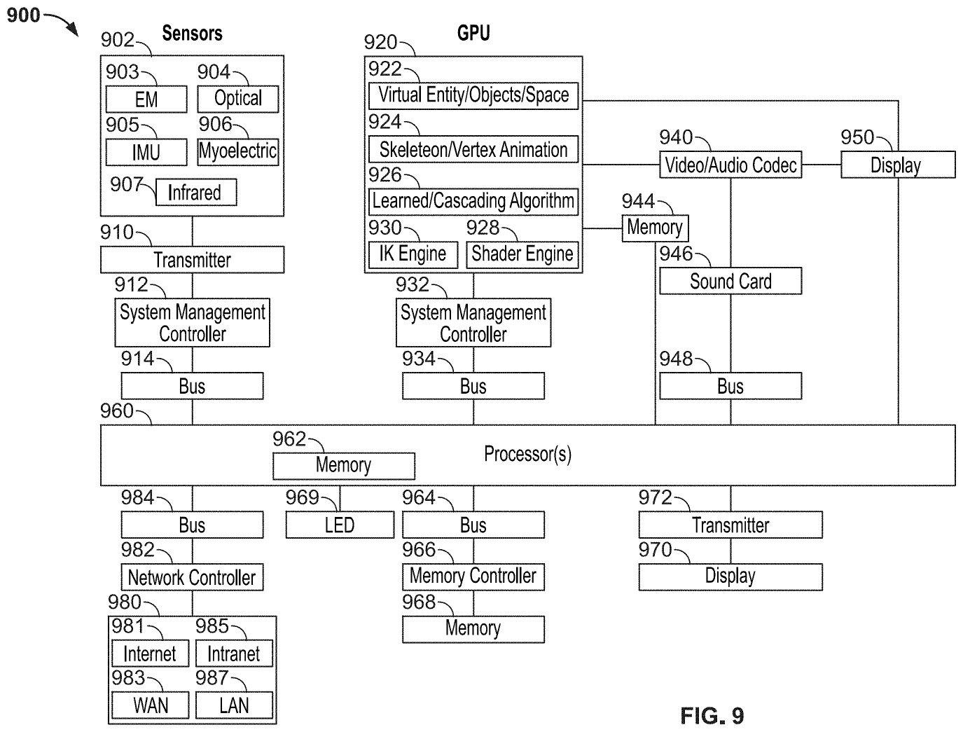

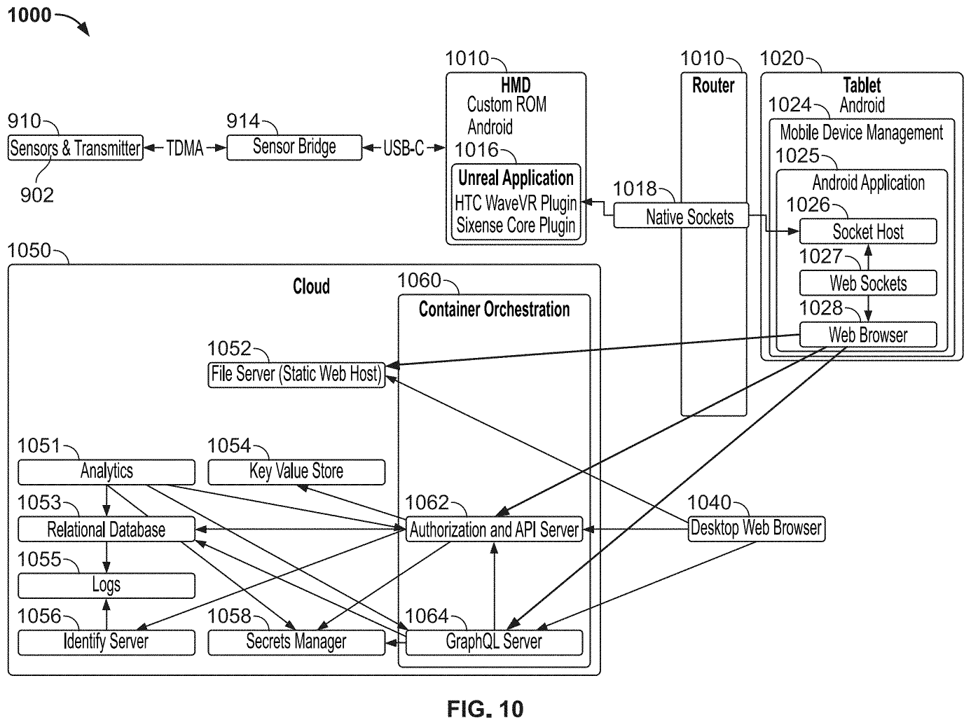

Patient View may be considered a view of the VR world from the VR headset. A VR environment rendering engine (sometimes referred to herein as a “VR application”) on a device, e.g., an HMD, such as the Unreal® Engine, may use the position and orientation data to generate a virtual world including an avatar that mimics the patient's movement and view. Unreal Engine is a software-development environment with a suite of developer tools designed for developers to build real-time 3D video games and applications, virtual and augmented reality graphics, immersive technology simulations, 3D videos, digital interface platforms, and other computer-generated graphics and worlds. A VR application may incorporate the Unreal Engine or another three-dimensional environment developing platform, e.g., sometimes referred to as a VR engine or a video game engine. Some embodiments may utilize a VR application, stored and executed by one or more of the processors and memory of a headset, server, tablet and/or other device to render scenario 100A. For instance, a VR engine may be incorporated in one or more of head-mounted display 201 and clinician tablet 210 of FIGS. 7A-D and/or the systems of FIGS. 9-10. A VR engine may run on a component of a tablet, HMD, server, display, television, set-top box, computer, smartphone, or other device. A VR engine may also generate an interface to display scenario 100A.

Spectator View, as seen, e.g., in scenario 100A, may be a copy of what the patient sees on the HMD while participating in a VR activity, e.g., Patient View. In some embodiments, Scenario 100A may be depicted on a therapist's tablet or display, such as clinician tablet 210 as depicted in FIG. 7A. For instance, a spectator, such as a therapist, may view scenario 140 and see a reproduction or mirror of a patient's view in the HMD, e.g., “Spectator View.” Spectator View may replicate a portion of the display presented to the patient, “Patient View,” that fits on a display, e.g., a supervisor tablet. Scenario 140 may be referred to as “Patient View” or “Spectator View.” In some embodiments, scenario 100A may be a carbon-copy reproduction of a portion of a Patient View from a participant's HMD, such as headset 201 of FIGS. 7A-D. In some embodiments, an HMD may generate a Patient View as a stereoscopic 3D image representing a first-person view of the virtual world with which the patient may interact. An HMD may transmit Patient View, or a non-stereoscopic version, as Spectator View to the clinician tablet for display. Spectator View may be derived from a single display, or a composite of both displays, from the stereoscopic Patient View.

In some embodiments, to generate and animate an avatar based on user movements, generally, a VR system collects raw sensor data (e.g., position, orientation, and acceleration data) from patient movements, filters the raw data, passes the filtered data to an inverse kinematics (IK) engine, and then an avatar solver may generate a skeleton and mesh in order to render and animate the patient's virtual avatar. An avatar includes virtual bones and comprises an internal anatomical structure that facilitates the formation of limbs and other body parts. To animate the avatar, an avatar solver may employ inverse kinematics and a series of local offsets to constrain the skeleton of the avatar to the position and orientation of the sensors. The VR skeleton then deforms a polygonal mesh to approximate the movement of the sensors. In some embodiments, systems and methods of the present disclosure may animate an avatar by blending the nearest key poses in proportion to their proximity to the user's tracked position, e.g., vertex animation. In some embodiments, a combination of vertex animation and skeletal animation may be applied to render an avatar. In some embodiments, movements of a movement library, such as those depicted in scenario 100A, may be mirrored for left-handed or right-handed individuals.

In some embodiments, for example, a tooth-brushing activity may be performed by a mocap performer wearing an HMD and sensors of FIGS. 7A-D with the VR system capturing movements while she brushes her teeth. Sensors may be placed on a body part in various positions, e.g., as depicted in FIG. 7C. During the capture, each sensor may transmit data, e.g., position and orientation (PnO) data, at a predetermined frequency, such as 200 Hz. For instance, data may include a position in the form of three-dimensional coordinates and rotational measures around each of three axes. In some embodiments, each sensor may include inertial measurement units (IMUs), described in FIG. 9, and may transmit acceleration data, as well as PnO data. Based on the sensor data, the VR system is able to generate avatar skeletal data for rendering and animating a virtual avatar.

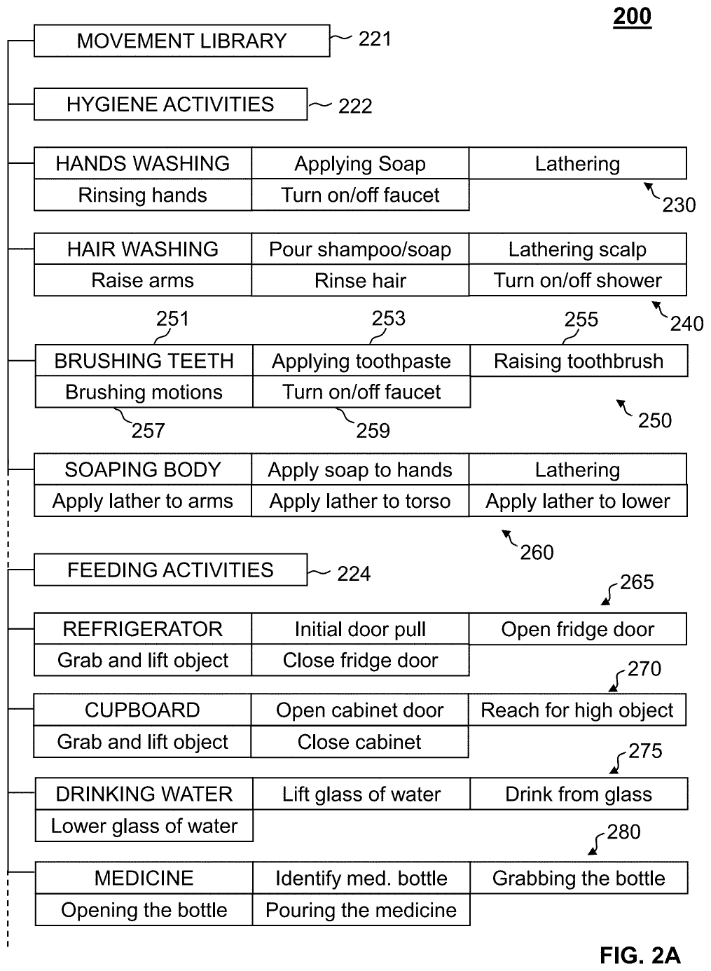

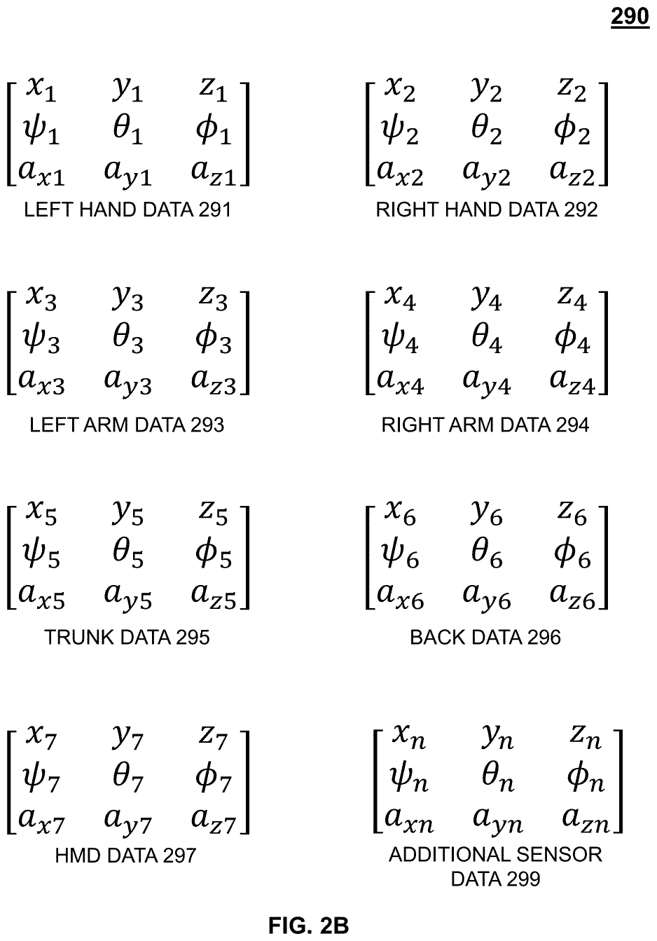

Sensor data may be construed as one or more matrices of, e.g., position, orientation, and/or acceleration data as depicted in FIG. 2B. In some embodiments, raw or filtered sensor data may be received and stored. In some embodiments, virtual skeletal data based on sensor data may be stored. Sensor data and/or skeletal data may be stored with profile and/or application data, e.g., in a database, e.g., as depicted in FIG. 10.

As the mocap performer brushes her teeth, while wearing an HMD and sensors, the VR system may generate avatar skeletal data based on sensor data and may render and animate a virtual avatar mimicking her movements. This animation may be recorded to a movement library so that someone may access the library and view movements at a later time. In some embodiments, the VR system may render and animate a virtual avatar at a later time, e.g., when providing the movement to the movement library. The movement library may be stored with profile and/or application data, e.g., in a database, e.g., as depicted in FIG. 10.

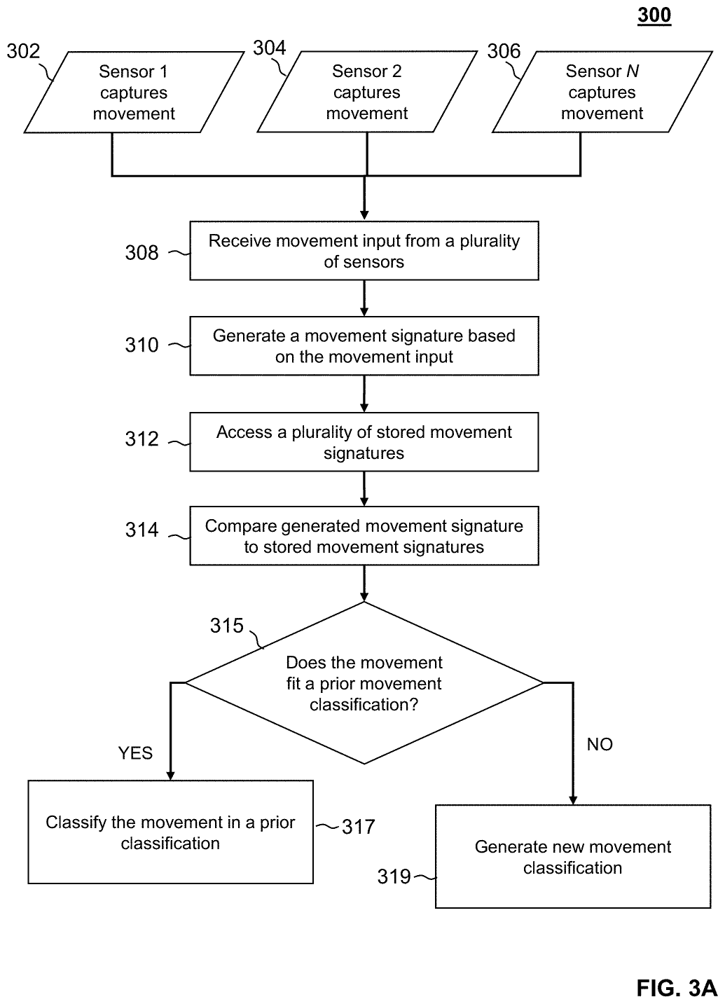

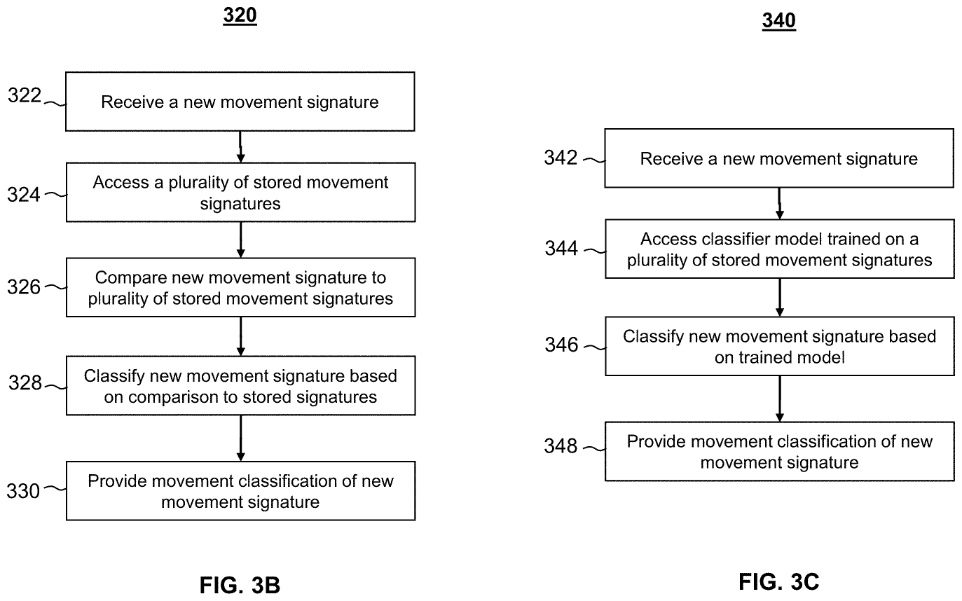

In scenario 100A, exemplary teeth brushing signature 110 corresponds to the teeth-brushing movement(s) depicted by the avatar illustration. For instance, a movement signature may be derived from captured sensor data to designate the movement. A movement signature may be generated based on plotting one or more of position, rotation, and acceleration, for one or more sensors, on a chart against another variable, e.g., time. For instance, for each unit of time, each of several sensors may produce a matrix of position, orientation, and acceleration data (e.g., as depicted in FIG. 2B) that may be plotted to produce a graph. Teeth brushing signature 110 of scenario 100A may be considered a representation of a corresponding movement signature based on changes of a hand on a vertical y-axis over time, which is not necessarily drawn to scale. Some embodiments may utilize a movement signature represented by a graph with many measurements (e.g., height, width, depth, pitch, roll, yaw, and acceleration in three directions) over a period of time, from a plurality of sensors (e.g., back, trunk, left and right hands, left and right arms, head, legs, knees, feet etc.). In some embodiments, a movement signature may be generated based on collecting one or more of position, rotation, and acceleration data for a specific sensor or body part in a data structure with a corresponding time. Some embodiments may classify a movement signature using a process such as processes 300, 320, and 340 depicted in FIGS. 3A-3C. Some embodiments may add a movement to a movement library using a process such as process 400 FIG. 4A.

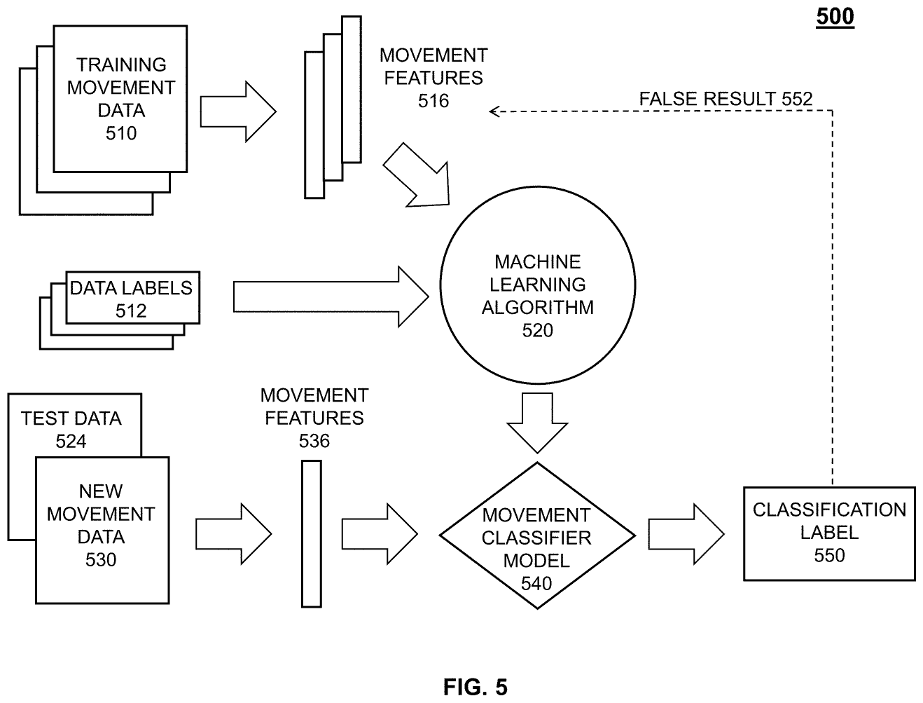

In some embodiments, a movement may be classified using a machine learning algorithm based on a movement library. For instance, a trained model may receive input of new movement data (e.g., a movement signature) and output an appropriate movement classification. In some embodiments, a movement classifier model labels any movement that is input as a particular activity. In some embodiments, a movement classifier model is trained on (a portion of) a movement library. FIG. 5 depicts an exemplary flow diagram of a process based in machine learning that may be used to train a movement classifier model. Generally, a movement classifier model may be trained with training movement data and a corresponding movement label. For instance, a VR system may capture known movements by mocap performers to create training data. Known movements may be, for example, brushing one's teeth (as depicted in FIG. 1A), washing one's hands (as depicted in FIG. 1B), and opening a refrigerator (as depicted in FIG. 1C). Once the movement classifier model is trained, the model may be given movement data and will produce a classification of the type of movement. In some embodiments, a model may be trained from a portion of a movement library and the movement library may grow from future movement captures and machine-learning based classifications. Some embodiments may classify a movement, e.g., using a process such as processes 300, 320, and 340 depicted in FIGS. 3A-3C. In some embodiments, a machine learning algorithm may cluster movement data and develop classifications of movements with similar qualities.

Depicted in scenario 100A, avatar 111 performs at least two smaller movements (e.g., micromovements) including raising action 112 and brushing action 114. For instance, avatar 111 may be displayed as raising a toothbrush and brushing his or her teeth in separate steps. In some embodiments, micromovements may be incorporated in one movement signature. In some embodiments, micromovements, such as raising action 112 and brushing action 114, may each have their own movement signature. In some embodiments, micromovements may be identified within a movement signature. For instance, a VR application may identify breaks, changes in direction, changes in rotation, changes in acceleration, changes in angular acceleration, etc. of one or more body parts based on a movement signature and extract one or more micromovements based on the identified breaks. FIG. 4B depicts an exemplary process of extracting micromovements from a movement. Looking at teeth brushing signature 110 of scenario 100A, featuring a graph of a y-position of a hand sensor, raising action 112 appears to correlate to a long, steep part of the curve and brushing action 114 appears to match an oscillating portion of the curve.

An activity of daily living, such as teeth brushing depicted in scenario 100A, may involve movements that could prove difficult for some people experiencing one or more various impairments. VR applications may be incorporate therapeutic activities to help patients learn, relearn, strengthen, and control body parts to better perform ADLs they may have difficulty with. In some embodiments, a VR application may directly ask a patient to perform a movement from an ADL as an exercise. For instance, a VR activity may display avatar 111 brushing his or her teeth and ask a patient to mimic the movements of the avatar, e.g., step by step. In some embodiments, such an activity may be used for instructions for or evaluation of a therapy patient.

In some embodiments, a VR application may indirectly ask a patient to perform a micromovement from an ADL as an exercise. For instance, a VR activity may require movement that incorporates a micromovement from a specific ADL, e.g., brushing one's teeth as depicted in scenario 100A. In some embodiments, for example, a VR activity involving feeding a virtual bird with a spoon may be used to encourage motions similar to raising a toothbrush, e.g., raising action 112 depicted in scenario 100A. In some embodiments, a VR activity involving painting shapes like circles on a virtual canvas may be used to help exercise a patient's hand dexterity and control to help practice motions like brushing teeth, e.g., brushing action 114 depicted in scenario 100A. In some embodiments, a patient's movement signature for each session may be compared to an exemplary movement signature to identify improvement (or deterioration) of the patient's movements and his or her ability to perform everyday activities. For instance, if a patient's movement signature gets closer (e.g., better match) to the mocap movement signature, it may be a sign of improvement, development, and/or strengthening.

In some embodiments, the VR platform can capture movements and micromovements of a patient, e.g., within VR activities, and compare them to one or more exemplary movement signatures to identify potentially problematic ADLs and activities. For instance, in a VR activity involving feeding a virtual bird with a spoon, capturing a patient's micromovement of lifting the spoon may allow a comparison to a movement signature from motion capture in the movement library, which may reveal problems with the patient's movement, e.g., lifting a toothbrush, lifting a hairbrush, and/or raising a fork. In some embodiments, potential motion problems may be associated with each VR activity. In some embodiments, a movement library can help diagnose a patient with potential issues, e.g., based on their prior motions in VR activity, as well as identify and provide VR activities that may help a patient practice and exercise particular motions to help with problematic movements.

FIG. 1B is an illustrative depiction of a captured movement and a movement signature for a VR therapy platform, in accordance with some embodiments of the disclosure. Scenario 100B of FIG. 1B depicts, e.g., a VR avatar washing his hands. Like many other hygiene activities, hand washing may be considered an important activity for independent living, as well as an ADL. Depicted in scenario 100B, avatar 121 performs at least three smaller movements (e.g., micromovements) including lather action 122, lowering action 124, and rinsing action 126.

Movements such as hand washing may be incorporated into the VR system, e.g., as part of a movement library. In some embodiments, a movement library may include data describing proper motions for various ADLs and activities. In some embodiments, each movement of a movement library may have a movement signature. A movement library may comprise a user interface that provides an avatar animation of an ADL, e.g., scenario 100B. In some embodiments, scenario 100B may be a portion of a VR application or activity used to help train patients. Some embodiments may add a movement to a movement library through a process such as the steps depicted in FIG. 4A. In some embodiments, movements of a movement library, such as those depicted in scenario 100B, may be mirrored for left-handed or right-handed individuals.

Scenario 100B of FIG. 1B may be considered a depiction of movements required to properly complete an ADL, e.g., hand washing, as captured by sensors positioned on a user of a VR system, e.g., a mocap performer. In some embodiments, for example, a handwashing activity may be performed by a mocap performer wearing an HMD and sensors of FIGS. 7A-D with the VR system capturing movements while he washes his hands. Sensors may be placed on a body part in various positions, e.g., as depicted in FIG. 7C. During the capture, each sensor may transmit sensor data, e.g., as one or more matrices of, e.g., position, orientation, and/or acceleration data as depicted in FIG. 2B.

As the mocap performer washes his hands, while wearing an HMD and sensors, the VR system may generate avatar skeletal data based on sensor data and may render and animate a virtual avatar mimicking her movements.

In scenario 100B, exemplary hand washing signature 120 corresponds to the hand washing movement(s) depicted by the avatar illustration. For instance, a movement signature may be derived from captured sensor data to designate the movement. Hand washing signature 120 of scenario 100B may be considered a representation of a corresponding movement signature based on changes of a hand on a vertical y-axis over time, which is not necessarily drawn to scale.

Depicted in scenario 100B, avatar 121 performs at least three smaller movements (e.g., micromovements) including lather action 122, lowering action 124, and rinsing action 126. For instance, avatar 121 may be displayed as soaping his hands, lowering his hands under a faucet, and rinsing his hands in separate steps. In some embodiments, micromovements may be incorporated in one movement signature. In some embodiments, micromovements, such as lather action 122, lowering action 124, and rinsing action 126, may each have their own movement signature. In some embodiments, micromovements may be identified within a movement signature. Looking at hand washing signature 120 of scenario 100B, featuring a graph of a y-position of a hand sensor, lather action 122 appears to match an initial oscillating portion of the curve, lowering action 124 appears to correlate to a long, steep part of the curve, and rinsing motion 126 appears to match a latter oscillating portion of the curve. An alternative depiction or illustration of these micromovement may involve diagrams of velocity vectors to show the incremental mentions or displacements that make up a movement signature.

An activity of daily living, such as hand washing depicted in scenario 100B, may involve movements that could prove difficult for some people experiencing one or more various impairments. VR applications may be incorporate therapeutic activities to help patients exercise certain movements with which they may have difficulty. In some embodiments, a VR activity may display avatar 121 washing his hands and ask a patient to mimic the movements of the avatar, e.g., step by step. In some embodiments, such an activity may be used for instructions for or evaluation of a therapy patient.

In some embodiments, a VR activity may require movement that incorporates a micromovement from a specific ADL, e.g., washing one's hands as depicted in scenario 100B. In some embodiments, for example, a VR activity involving rubbing a stick to start a fire, e.g., at different speeds, may be used to encourage motions similar to lathering and/or rinsing one's hands, e.g., lather action 122 and rinsing action 126 depicted in scenario 100B. In some embodiments, a VR activity involving lifting and lowering a bird using both hands may be used to help exercise a patient's arm dexterity and control to help practice motions like lowering hands below a faucet, e.g., lowering action 124 depicted in scenario 100B.

In some embodiments, the VR platform may identify potentially problematic ADLs and activities based on comparing captured movements/micromovements of a patient, e.g., within VR activities, with one or more exemplary movement signatures from a movement library. For instance, in a VR activity involving rubbing a stick to start a fire, capturing a patient's micromovement of rubbing sticks together may allow a comparison to a movement signature from motion capture in the movement library, which may reveal problems with the patient's movement, e.g., lathering soap, drying hands, and/or coordinating hand motions.

FIG. 1C is an illustrative depiction of a captured movement and a movement signature for a VR therapy platform, in accordance with some embodiments of the disclosure. Scenario 100C of FIG. 1C depicts, e.g., a VR avatar opening a refrigerator. Like many other kitchen-area activities, opening a refrigerator may be considered an important activity for independent living, as well as an ADL. Depicted in scenario 100C, avatar 131 performs at least two smaller movements (e.g., micromovements) including jerk action 132 and pulling action 134.

Movements related to opening a refrigerator may be incorporated into the VR system, e.g., as part of a movement library. In some embodiments, a movement library may include data describing proper motions for various ADLs and activities. In some embodiments, each movement of a movement library may have a movement signature. A movement library may comprise a user interface that provides an avatar animation of an ADL, e.g., scenario 100C. In some embodiments, scenario 100C may be a portion of a VR application or activity used to help train patients. Some embodiments may add a movement to a movement library through a process such as the steps depicted in FIG. 4A. In some embodiments, movements of a movement library, such as those depicted in scenario 100C, may be mirrored for left-handed or right-handed individuals.

Scenario 100C of FIG. 1C may be considered a depiction of movements required to properly complete an ADL, e.g., refrigerator opening, as captured by sensors positioned on a user of a VR system, e.g., a mocap performer. In some embodiments, for example, a refrigerator-opening activity may be performed by a mocap performer wearing an HMD and sensors of FIGS. 7A-D with the VR system capturing movements while he opens a refrigerator. Sensors may be placed on a body part in various positions, e.g., as depicted in FIG. 7C. During the capture, each sensor may transmit sensor data, e.g., as one or more matrices of, e.g., position, orientation, and/or acceleration data as depicted in FIG. 2B.

As the mocap performer opens a refrigerator door, while wearing an HMD and sensors, the VR system may generate avatar skeletal data based on sensor data and may render and animate a virtual avatar mimicking her movements.

In scenario 100C, exemplary refrigerator opening signature 130 corresponds to the refrigerator opening movement(s) depicted by the avatar illustration. For instance, a movement signature may be derived from captured sensor data to designate the movement. Refrigerator opening signature 130 of scenario 100C may be considered a representation of a corresponding movement signature based on changes of a hand on a horizontal z-axis over time, which is not necessarily drawn to scale.

Depicted in scenario 100C, avatar 131 performs at least two smaller movements (e.g., micromovements) including jerk action 132 and pulling action 134. For instance, avatar 131 may be displayed as jerking the refrigerator door open and gently pulling the refrigerator door further open in separate steps. In some embodiments, micromovements may be incorporated in one movement signature. In some embodiments, micromovements, such as jerk action 132 and pulling action 134, may each have their own movement signature. In some embodiments, micromovements may be identified within a movement signature. Looking at refrigerator opening signature 130 of scenario 100C, featuring a graph of a z-position of a hand sensor, jerk action 132 appears to match an initial steep part of the curve and pulling action 134 appears to correlate to a later less-steep part of the curve.

An activity of daily living, such as opening a refrigerator depicted in scenario 100C, may involve movements that could prove difficult for some people experiencing one or more various impairments. VR applications may be incorporate therapeutic activities to help patients exercise certain movements with which they may have difficulty. In some embodiments, a VR activity may display avatar 131 opening a refrigerator and ask a patient to mimic the movements of the avatar, e.g., step by step. In some embodiments, such an activity may be used for instructions for or evaluation of a therapy patient.

In some embodiments, a VR activity may require movement that incorporates a micromovement from a specific ADL, e.g., opening a refrigerator as depicted in scenario 100C. In some embodiments, for example, a VR activity involving quickly pulling a rope, e.g., in a tug of war, may be used to encourage motions similar to jerking a door from a vacuum seal, e.g., jerk action 132 depicted in scenario 100C. In some embodiments, a VR activity involving opening doors as part of a hide-and-seek with woodland critters may be used to help exercise a patient's arm dexterity and control to help practice motions like gently pulling a door open, e.g., pulling action 134 depicted in scenario 100C.

In some embodiments, the VR platform may identify potentially problematic ADLs and activities based on comparing captured movements/micromovements of a patient, e.g., within VR activities, with one or more exemplary movement signatures from a movement library. For instance, in a VR activity involving pulling a rope, capturing a patient's micromovement of pulling quickly may allow a comparison to a movement signature from motion capture in the movement library, which may reveal problems with the patient's movement, e.g., opening doors, opening refrigerators, turning on faucets, pulling luggage and/or grabbing and retrieving objects.

FIG. 1D is an illustrative depiction of a user interface for a VR therapy platform, in accordance with some embodiments of the disclosure. Scenario 140 of FIG. 1D illustrates a user interface of a virtual reality application that may be delivered via Patient View, Spectator View, or via another interface in a VR application, e.g., exploring a movement library. Scenario 140 may be displayed to a patient view via the head-mounted display, e.g., “Patient View.” Scenario 140 may also be considered a user interface of the same VR application as depicted to a spectator, such as a therapist.

Interface 150 of scenario 140 of FIG. 1D may be considered a menu for a VR therapy platform. In some embodiments, interface 150 depicts suggested VR activities 172, 174, 176, and 178 based on an identified patient profile 152. Each of profile 152 and VR activities 172, 174, 176, and 178 based on patient profile 152 may be presented with a representative image or icon. Each of profile 152 and VR activities 172, 174, 176, and 178 based on patient profile 152 may be presented with descriptions, e.g., movements that may be exercised.

Interface 150 depicts patient profile 152 for “Jane Doe.” Patient profile 152 is shown to be documented with the patient having ADL goals of, e.g., teeth brushing, hand washing, and refrigerator opening. Patient profile 152 may include further impairment data, health data, VR activity data, and other relevant data. Patient profile 152 may be accessed and loaded, e.g., as a patient logs in to interface 150, e.g., the VR therapy platform. In some embodiments, loading patient profile 152 may be initiated by a therapist or supervisor.

Interface 150 further depicts VR activities 172, 174, 176, and 178. In some embodiments, VR activities 172, 174, 176, and 178 may be, e.g., applications, environments, activities, games, characters, sub-activities, tasks, videos, and other content. In scenario 140 of FIG. 1D, in this example, VR activity 172 represents an activity titled “Feed the Squirrels.” VR activity 172 is depicted as promoting motion to “[l]ift the spoon up and down to feed the squirrels.” In some embodiments, lifting a spoon to feed a squirrel may correspond to a micromovement involved in lifting a toothbrush for brushing one's teeth. In scenario 140 of FIG. 1D, VR activity 174 represents a VR activity titled “Start the Campfire.” VR activity 174 is depicted as promoting movement where a patient “[r]ubs the sticks together to make a campfire.” In some embodiments, rubbing a stick may correspond to a micromovement used in washing one's hands, e.g., building lather and/or rinsing. VR activity 176 may be considered to represent an activity titled “Grab the Fruit.” VR activity 176 is depicted as promoting movement by asking the patient to “[g]rab a fruit you see and squeeze each fruit.” In some embodiments, grabbing may be considered a micromovement involved in, e.g., grabbing food from a refrigerator. In some embodiments, squeezing may be considered a micromovement involved in putting toothpaste on a toothbrush. In scenario 140, VR activity 178 represents a VR activity titled “Find the Woodland Friends.” VR activity 178 is depicted as requiring a patient to “[o]pen doors to find the animals.” In some embodiments, opening a door may be considered a micromovement involved in, e.g., opening a refrigerator door.

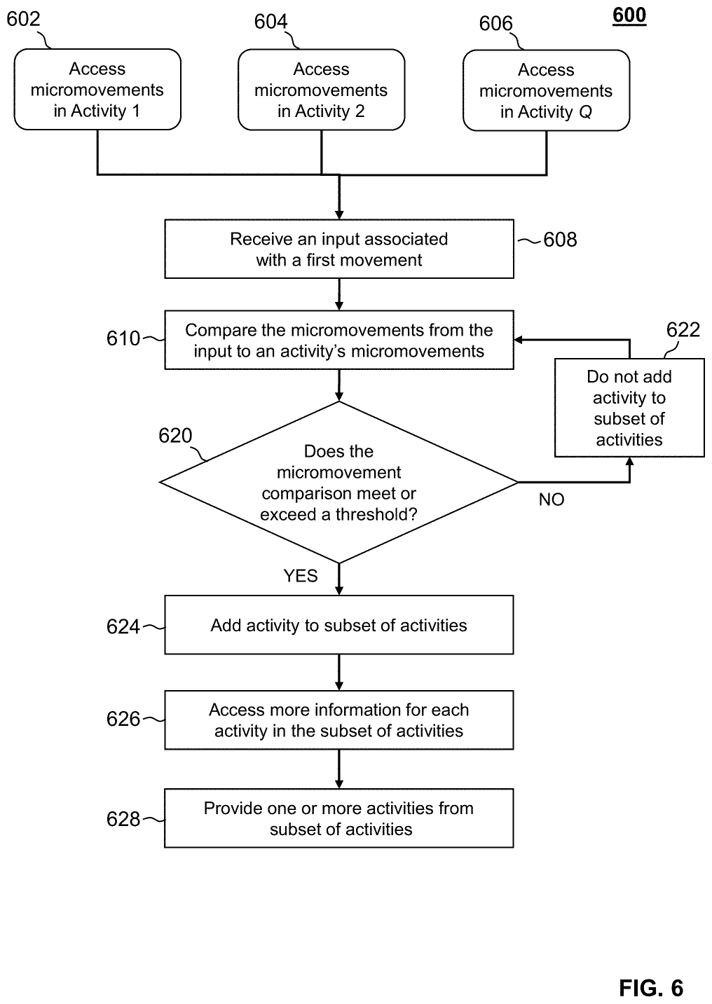

In some embodiments, VR activities 172, 174, 176, and 178 may be selected as suggested or recommended for patient profile 152. For instance, interface 150 may analyze impairments of patient profile 152 and impairments of each of the VR activities/exercises in the system to determine which activities would be most appropriate for the patient. Selecting activities to present may be accomplished in several ways. Process 600 of FIG. 6 is an exemplary process for selecting one or more activities, e.g., based on a desire for a patient to perform certain movements or micromovements.

In some embodiments, the VR platform may identify potentially problematic ADLs and activities based on comparing captured movements/micromovements of a patient, e.g., within VR activities, with one or more exemplary movement signatures from a movement library. For instance, required movements in “Feed the Squirrels” may reveal problems with the patient's movement, e.g., lifting utensils, brushing teeth, and/or steadily coordinating hand movement. In some embodiments, a VR activity “Start the Campfire” may allow a comparison to a movement signature from motion capture in the movement library and reveal problems with the patient's movement, e.g., lathering soap, drying hands, and/or coordinating hand motions. In some embodiments, a VR activity requiring squeezing fruit in, e.g., “Grab the Fruit,” may reveal problems with the patient's movement, e.g., grabbing items, squeezing items, using doorknobs and handles, and/or grabbing and retrieving objects. In some embodiments, a VR activity requiring opening doors to find animals in, e.g., “Find the Woodland Friends,” may reveal problems with the patient's movement, e.g., opening doors, opening refrigerators, turning on faucets, pulling luggage and/or grabbing and retrieving objects.

FIG. 2A depicts an illustrative data structure for a movement library, in accordance with some embodiments of the disclosure. Data structure 200 is an exemplary movement library data structure for recording movements (and micromovements) for organization and comparison in treating patients via VR activities. In some embodiments, a movement library data structure may comprise a hierarchical data structure, trees, linked lists, queue, playlists, matrices, tables, blockchains, and/or various other data structures. A movement library data structure may include, for instance, several levels of activity categories, ADLs, IADLs, activities, movements, micromovements, impairments, diagnoses, conditions, and linkages among items. Movement library 112 may include fields for activity categories, ADLs, IADLs, activities, movements, micromovements, impairments, diagnoses, conditions, and linkages.

Movement library data structure 200 depicts movement library 221. Movement library 221 includes activity categories such as hygiene activities 222 and kitchen activities 224. In some embodiments, movement library 221 may include categories for activities, e.g., based on importance to independent living. Within each category are activities 230, 240, 250, 260, 265, 270, 275, and 280. For instance, hygiene activities 222 includes activity 230, “hands washing,” activity 240, “hair washing,” activity 250, “brushing teeth,” and activity 260, “soaping body,” among others. Kitchen activities 224 includes activity 265, “refrigerator opening,” activity 270, “cupboard opening,” activity 275, “drinking a glass of water,” and activity 280, “taking medicine,” among others.

In some embodiments, a movement library data structure may include movements and smaller movements, e.g., sub-movements or micromovements, that may be required to carry out an activity. Movement library 221 includes micromovements linked with each movement of an activity. For instance, activity 250 is labeled with movement 251, “Brushing Teeth,” and includes micromovements such as, micromovement 253, “Applying toothpaste,” micromovement 255, “Raising toothbrush,” micromovement 257, “Brushing motions,” and micromovement 259, “Turn on/off faucet.” Of course, each activity may have several micromovements associated with it. For example, teeth brushing may also involve gripping a toothbrush, squeezing toothpaste, rinsing a toothbrush, rinsing one's mouth out, and various brushing motions, as well as spitting and some movements which may or may not be practical to practice via VR system. In some embodiments, micromovements may be identified within a movement signature, e.g., by plotting sensor data versus time. For instance, a VR application may identify breaks, changes in direction, changes in rotation, changes in acceleration, changes in angular acceleration, etc. of one or more body parts based on a movement signature and extract one or more micromovements based on the identified breaks. FIG. 4B depicts an exemplary process of extracting micromovements from a movement.

Some micromovements may be shared among several activities. For instance, some general micromovements like turning on or off a faucet may be shared among hand washing, teeth brushing, shampooing/showering, drinking water, and more. Likewise, micromovements for raising one's arms for hair washing and reaching for a high object from a kitchen cupboard may be very similar and may invoke similar VR activities to help practice. Even within activity 230, “hand washing,” micromovements of lathering one's hands and rinsing one's hands are very similar and the motions may be practiced together within one VR activity, at times.

In some embodiments, movement library 112 may include micromovements like “applying soap,” “lathering,” “rinsing hands,” and “tuning on/off a faucet,” among others, for activity 230, “hands washing.” In some embodiments, movement library 112 may include micromovements like “pour shampoo/soap,” “lathering scalp,” “arm raising,” “rinse hair,” and “tuning on/off the shower,” among others, for activity 240, “hair washing.” In some embodiments, movement library 112 may include micromovements such as “initial door pull,” “open fridge door,” “grab and lift object,” “close fridge door,” among others, for activity 265, “refrigerator opening.” In some embodiments, movement library 112 may include micromovements such as “lifting a glass of water,” “drinking from the glass,” “lowering a glass of water,” among others, for activity 275, “drinking a glass of water.” In some embodiments, movement library 112 may include micromovements like “identifying a (correct) medicine bottle,” “grabbing the bottle,” “opening the bottle,” “pouring the medicine,” among others, for activity 280, “taking medicine,” which may be considered an IADL.

Identifying micromovements associated with movements and activities may be valuable when selecting VR therapy activities to help strengthen and improve micromovements. In some embodiments, a movement library data structure may include links between activities that may allow a patient to practice micromovements associated with an ADL, e.g., activity 230, “hands washing.” For instance, activity 174, “Start the Campfire,” of FIG. 1D indicates that rubbing sticks to make a campfire may be a helpful exercise for lathering and/or rinsing hands. Likewise, a link to activity 178 of FIG. 1D, “Find the Woodland Friends,” may be included with activity 265, “refrigerator opening,” as the VR activity aims to practice “opening doors to find animals.”

In some embodiments, a movement library data structure may be stored in or with application and activity data in a database, e.g., as depicted in FIG. 10. In some embodiments, a movement library data structure may be stored, for instance, at an encrypted cloud server. Moreover, in some embodiments, a movement library data structure may be stored locally at the device.

FIG. 2B depicts illustrative data structures for movement data, in accordance with some embodiments of the disclosure. Data collection 290 of FIG. 2B depicts, for instance, a collection of sensor data, e.g., from at last seven different sensors. In some embodiments, each sensor may measure, at least, position, rotation, and acceleration (e.g., using IMUs, as identified in FIG. 9). Sensors may be placed on a body part in various positions, e.g., as depicted in FIG. 7C. Sensor data in data collection 290 signifies data collected as left hand data 291, right hand data 292, left arm data 293, right arm data 294, trunk data 295, back data 296, HMD data 297, and any additional sensor data 299. Some embodiments may incorporate more or fewer sensors.

At any a given moment in time, each sensor may capture and transmit sensor data including a position, e.g., in the form of three-dimensional coordinates, a rotational measurement around each of the three axes, and/or three dimensions of acceleration data. In some embodiments, each sensor may transmit sensor data at a predetermined frequency, such as 200 Hz. In some embodiments, each sensor may capture and transmit sensor data at different intervals, such as 15-120 times per second.

FIG. 2B depicts seven matrices, each with variables indicating measurements of position, rotation, and acceleration. For instance, left hand data matrix 291 includes a position on the x-axis, x₁, a position on they-axis, y₁, and a position on the z-axis, z₁. In some embodiments, a sensor data matrix, such as left hand data matrix 291, may further include rotational measurements of yaw, pitch, and roll, e.g., ψ₁, θ₁, and φ₁. In some embodiments, a sensor data matrix, such as left hand data matrix 291, may include acceleration measurements in each axial direction, e.g., αₓ₁, α_{y1}, and α_{z1}. In some embodiments, a sensor data matrix may be separated into multiple matrices. In some embodiments, multiple sensor data matrices may be combined into one or more matrices.

FIG. 3A depicts an illustrative flowchart of a process for classifying a movement, in accordance with some embodiments of the disclosure. There are many ways to identify appropriate VR activities and sub-activities for treating a patient and process 300 is one example. Generally, process 300 of FIG. 3A includes steps for receiving movement input (e.g., from sensors), generating a movement signature, and determining a movement classification.

Some embodiments may utilize a VR engine to perform one or more parts of process 300, e.g., as part of a VR application, stored and executed by one or more of the processors and memory of a headset, server, tablet and/or other device. For instance, a VR engine may be incorporated in one or more of head-mounted display 201 and clinician tablet 210 of FIGS. 7A-D and/or the systems of FIGS. 9-10. A VR engine may run on a component of a tablet, HMD, server, display, television, set-top box, computer, smartphone, or other device.

At step 302, a first sensor captures a movement performed by a user. A first sensor may be one of a plurality of sensors placed on a user's body, e.g., as depicted in FIG. 7C. For instance, in a motion such as teeth brushing, as depicted in FIG. 1A, a right hand sensor may be considered a first sensor. In some embodiments, only one sensor may be used. For instance, in some cases, measuring motion of one hand may be sufficient to measure and identify a movement.

At step 304, a second sensor captures the movement performed by a user. A second sensor may be one of a plurality of sensors placed on a user's body, e.g., as depicted in FIG. 7C. For instance, in a motion such as teeth brushing, as depicted in FIG. 1A, a right arm sensor may be considered a second sensor.

At step 306, an N sensor captures the movement performed by a user. An N sensor may be one of a plurality of sensors placed on a user's body, e.g., as depicted in FIG. 7C. For instance, in a motion such as teeth brushing, as depicted in FIG. 1A, a head, back, or trunk sensor may be considered a second sensor. In some embodiments, N may any number greater than two. In some embodiments, such as sensor data depicted in FIG. 2B and sensor positions in FIG. 7C, there may be seven or more sensors, including, e.g., left hand, right hand, left arm, right arm, trunk, back, and HMD.

At step 308, the VR engine receives movement input from a plurality of sensors. Generally, each sensor may detect position and orientation (PnO) data and transmit such data to the HMD for processing. In some embodiments, each sensor may detect and transmit acceleration data e.g., based on inertial measurement units (IMUs), described in FIG. 9. In some embodiments, measured acceleration data may be used to improve accuracy of PnO data.

At step 310, the VR engine generates a movement signature based on the movement input. Generally, generating a movement signature comprises generating a visual or mathematical representation of data describing a movement. For instance, a movement signature may be a graph of sensor data over time. In some embodiments, generating a movement signature may include steps of plotting one or more portions of sensor data as graphs and generating a movement signature based on the graphs. Process 400 of FIG. 4A describes an exemplary process for generating a movement signature and adding the movement signature to a movement library. In some embodiments, a movement signature may comprise normalized values of data describing one or more movements. In some embodiments, a movement signature may comprise values in one or more matrices or other data structures. In some embodiments, a movement signature may comprise a movement data expressed as a wave. In some embodiments, a movement signature may comprise movement data expressed as a function and/or equation. In some embodiments, a movement signature may comprise VR avatar skeletal movement data based on the sensor data. In some embodiments, a movement signature may comprise VR avatar animation data and/or images.

At step 312, the VR engine accesses a plurality of stored movement signatures. In some embodiments, a movement library may store a plurality of movement signatures. For instance, movement library 221 in data structure 200 of FIG. 2A depicts an exemplary movement library with a plurality of movements, e.g., each associated with a movement signature. In some embodiments, a movement library may include movements related to one or more activities. For instance, a movement library may include an activities hands washing, hair washing, brushing teeth, soaping body, refrigerator opening, cupboard opening, drinking a glass of water, and taking medicine, among others. Each activity may be associated with a movement signature that may be used to identify similar movements and classify as such. In some embodiments, a movement library data structure may include movements and smaller movements, e.g., sub-movements or micromovements, that may be required to carry out an activity. In some embodiments, a movement library may be incorporated as part of a trained classifying model using, e.g., machine learning. In some embodiments, movements may be compared to prior movement data for the patient, e.g., in order to determine improvement or debilitation, in addition to classifying movements.

At step 314, the VR engine compares the generated movement signature to stored movement signatures. Generally, the VR engine may compare the generated movement signature to the stored movement signatures and classify the movement signature based on the comparison. In some embodiments, the generated movement signature may be compared to each of the stored movement signatures and a movement match score may be determined. For instance, process 320 of FIG. 3B depicts exemplary steps for comparing movement signatures. In some embodiments, a movement match score may be determined based on correlation of the generated movement signature with each of the stored movement signatures. For instance, a match score of 0-99 may be attributed to a comparison of two movement signatures based on, e.g., a measure of correlation. For example, a patient with a movement disorder may perform a hand washing motion and produce a motion signature that correlates to a motion-captured hand-washing signature at a movement match score of, e.g., 85. In some embodiments, a movement match score may be determined based on regression and/or other statistical comparisons. In some embodiments, a movement match score may be determined based on image analytics of graphic representations of movements. In some embodiments, a trained classifier model may perform comparisons. A classifier model may be trained similarly to training movement classifier model 540 of process 500 as depicted in the flow diagram of FIG. 5.

At step 315, the VR engine determines whether the movement fits a prior movement classification. For instance, in some embodiments, a movement match score may be determined based on the comparison between the generated movement signature and each of the stored movement signatures and the highest movement match score will determine the movement classification. In some embodiments, if the highest movement match score is not above a threshold, e.g., 75 on a scale of 0-99, then the highest movement match score may not be a good fit or sufficient match. For example, a patient with a movement disorder may perform a teeth-brushing motion and, due to tremors, may produce a movement match score of 77 with a motion capture teeth-brushing movement signature, which may be sufficient. In some cases, a patient rehabilitating a serious shoulder injury may perform a motion of washing his hair and may produce a movement match score of 65 with a motion capture movement signature, which may be insufficient to classify the patient's movement as hair washing. In some embodiments, other statistical analysis may be used to determine the highest ranked match score is or is not a good fit. In some embodiments, a trained model may determine if the best matching classification is a good enough fit or requires a new classification.

If, at step 315, the VR engine determines the movement fits a prior movement classification, at step 317, the VR engine classifies the movement in a prior classification. For instance, in some embodiments, if the highest movement match score is above a threshold, e.g., 76 on a scale of 0-99, then the highest movement match score may be deemed a good fit and/or a sufficient match. For example, a patient recovering from a stroke may perform a refrigerator-door-opening motion and may produce a movement match score of 78 with a motion capture movement signature, which may be sufficient to classify the patient's movement. In some embodiments, a trained model may determine if the best matching classification is a good enough fit for the classification.

If, at step 315, the VR engine determines the movement does not fit a prior movement classification, at step 319, the VR engine generates new movement classification. In some cases, the best match is not sufficient as a classification. For instance, in some embodiments, if the highest movement match score is below a threshold, e.g., 67 on a scale of 0-99, then the highest movement match score may be deemed a good fit and/or a sufficient match. For example, a motion capture actor may perform a motion for using hand sanitizer for her hands and may produce a movement match score of 63 with a motion capture movement signature for hand washing. Sanitizing one's hands may use very similar movements as hand washing, but if a match score is below a threshold, the motion signature may require a new classification. In some embodiments, a trained model may determine if the best matching classification is not a good enough fit and requires a new classification.

FIG. 3B depicts an illustrative flowchart of a process for classifying a movement signature, in accordance with some embodiments of the disclosure. There are many ways to classify a movement signature and process 320 is one example. Generally, process 320 of FIG. 3B includes steps for receiving a movement signature as input, accessing a plurality of movement signatures, comparing the input movement signature to the stored signatures, classifying the movement signature based on the comparison, and providing the movement classification.

Some embodiments may utilize a VR engine to perform one or more parts of process 320, e.g., as part of a VR application, stored and executed by one or more of the processors and memory of a headset, server, tablet and/or other device.

At step 322, a VR engine receives a new movement signature, e.g., as input. A movement signature may be considered a visual, graphical, or mathematical representation of data describing a movement. For instance, a movement signature may be a graph of sensor data, or VR avatar data, over time. Process 400 of FIG. 4A describes an exemplary process for generating a movement signature. In some embodiments, user data (e.g., a patient profile) may be input with movement data. For instance, a patient's height, weight, sex, age, body mass, and impairment and/or health history may affect a movement.

At step 324, the VR engine accesses a plurality of stored movement signatures. In some embodiments, a movement library may store a plurality of movement signatures. For instance, movement library 221 in data structure 200 of FIG. 2A depicts an exemplary movement library with several movement, e.g., each associated with a movement signature.

At step 326, the VR engine compares new movement signature to a plurality of stored movement signatures. In some embodiments, the generated movement signature may be compared to each of the stored movement signatures and a movement match score may be determined. In some embodiments, a movement match score may be determined based on correlation of the generated movement signature with each of the stored movement signatures. For instance, a match score of 0-99 may be attributed to a comparison of two movement signatures based on, e.g., a measure of correlation. For example, a patient with a movement disorder may perform a hand washing motion and produce a motion signature that correlates to a motion-captured hand-washing signature at a movement match score of, e.g., 85. In some embodiments, a movement match score may be determined based on regression and/or other statistical comparisons. In some embodiments, a movement match score may be determined based on image analytics of graphic representations of movements.

At step 328, the VR engine classifies new movement signature based on the comparison to the stored signatures. For instance, in some embodiments, a movement match score may be determined based on the comparison between the generated movement signature and each of the stored movement signatures and the highest movement match score will determine the movement classification. In some embodiments, a patient with a movement disorder may perform a teeth-brushing motion and may produce a movement match score of 77 (e.g., on a scale of 0-99), when compared to a motion capture teeth-brushing movement signature and may produce a movement match score of, e.g., 45 when compared to a movement signature based on motion capture of a trained person eating soup. In some cases, a patient rehabilitating a serious shoulder injury may perform a motion of washing his hair and may produce a movement match score of 65 with a motion capture movement signature and may produce a movement match score of 45 when compared to a motion capture hand-washing movement signature, e.g., due to his limited motion. In some embodiments, other statistical analysis may be used to determine the highest movement match score. In some embodiments, this may be based on a count of multiple high-ranking match scores. For instance, there may be several movement signatures for hand washing and hair washing—each derived from trained motion capture—and while one movement signature for hand washing may generate a high movement match score, there may be several highly ranked movement match scores for hair washing (e.g., four, above a threshold of three) that indicate a classification of hair washing rather than hand washing.

At step 330, the VR engine provides the movement classification of the input movement signature. For instance, the VR engine may output or relay a movement classification based on the input movement signature (or other movement data). In some embodiments this may be the highest movement match score. In some embodiments, this may be based on a count of multiple high-ranking match scores of similar classifications.

FIG. 3C depicts an illustrative flowchart of a process for classifying a movement signature, in accordance with some embodiments of the disclosure. There are many ways to classify a movement signature and process 340 is one example. Generally, process 340 of FIG. 3C includes steps for receiving a movement signature, accessing a model that was trained on a plurality of movement signatures, classifying the input movement signature using the trained model based on the stored signatures, and providing the movement classification.

Some embodiments may utilize a VR engine to perform one or more parts of process 340, e.g., as part of a VR application, stored and executed by one or more of the processors and memory of a headset, server, tablet and/or other device.

At step 342, the VR engine receives a new movement signature. A movement signature may be considered a visual or mathematical representation of data describing a movement. For instance, a movement signature may be a graph of sensor data, or VR avatar data, over time. Process 400 of FIG. 4A describes an exemplary process for generating a movement signature. In some embodiments, user data (e.g., a patient profile) may be input with movement data. For instance, a patient's height, weight, sex, age, body mass, and impairment and/or health history may affect a movement.

At step 344, the VR engine accesses a classifier model that was trained from a plurality of stored movement signatures. For instance, a classifier model may be trained similarly to training movement classifier model 540 of process 500 as depicted in the flow diagram of FIG. 5. In some embodiments, a classifier model may be trained from a plurality of previously generated and stored movements and/or their movement signatures. In some embodiments, a classifier model may be trained from training movement data comprising previously generated and stored sensor data and/or movement data. In some embodiments, a classifier model may use a movement library. For instance, movement library 221 in data structure 200 of FIG. 2A depicts an exemplary movement library with several movement, e.g., each associated with a movement signature.

At step 346, the VR engine classifies new movement signature based on the trained movement classifier model. For instance, patient motion may be input and classified as a particular activity or movement. Generally, in some embodiments, movement signature data may be processed to yield one or more movement features and passed to movement classifier model for a classification. In some embodiments, a trained movement classifier model may evaluate movement features and present a classification label of a movement and/or activity.

At step 348, the VR engine provides movement classification for the input movement signature. For instance, the movement classifier model may output the determined movement or activity classification label based on the input data, in light of training from prior movement data. If a classification label for new movement data can be verified outside the system, the movement classifier model may be further updated with feedback and reinforcement for further accuracy.

FIG. 4A depicts an illustrative flowchart of a process for adding a movement to a movement library, in accordance with some embodiments of the disclosure. There are many ways to add a movement to a movement library and process 400 is one example. Generally, process 400 of FIG. 4A includes steps for sensors capturing movement performed by a user (e.g., position, rotation, and acceleration), plotting one or more portions of sensor data as graphs, generating a movement signature based on the graphs, and adding the movement signature to the movement library. Some embodiments may utilize a VR engine to perform one or more parts of process 400, e.g., as part of a VR application, stored and executed by one or more of the processors and memory of a headset, server, tablet and/or other device.

In some embodiments, movements of process 400 may be performed by patients learning or re-learning how to perform vital activities, e.g., ADLs. In some cases, motion capture sessions may be performed by a motion-capture performer—working with, e.g., a doctor, therapist, VR application designer, supervised assistant, actor, or other trained professional— performing the activity, e.g., brushing teeth or drinking water from a glass, to capture proper form of the motion in a movement library. For instance, VR hardware and sensors may be positioned on the body of the mocap performer, and she may perform the necessary steps of the ADL for capture by the VR system and incorporation into a movement library. Sensors of the VR system may capture a performed movement and the VR system may translate real-world movement to movement by a VR avatar in a virtual world. Real-world performance may be completed with or without props, inside or outside the usual setting (e.g., bathroom, kitchen). In some embodiments, a mocap performer may be able to pantomime an ADL using movements proper enough for capture and training. Process 400 may be performed on movement data generated by a patient or a mocap actor alike.

At step 402, a plurality of sensors captures position data of movement performed by a user. For instance, each sensor may capture position data for three axes. In some embodiments, for example, a position data matrix for sensor “n” may include a position on the x-axis, xₙ, a position on the y-axis, yₙ, and a position on the z-axis, zₙ.

At step 404, a plurality of sensors captures position data of movement performed by a user. For instance, each sensor may capture orientation and/or rotational data about three axes. In some embodiments, a sensor data matrix may include rotational measurements of yaw, pitch, and roll, e.g., ψ, θ, and φ.

At step 406, a plurality of sensors captures position data of movement performed by a user. For instance, each sensor may capture IMU and/or acceleration data for three axes. In some embodiments, each sensor may include IMU data inertial measurement units (IMUs), described in FIG. 9, and may measure and transmit acceleration data. In some embodiments, a sensor data matrix may include acceleration measurements in each axial direction, e.g., αₓₙ, α_{yn}, and α_{zn}.

At step 410, the VR engine receives sensor data (e.g., position, orientation, and/or acceleration). For example, a receiver connected to the VR engine may receive signals transmitted by each sensor. In some embodiments, sensor data may be construed as one or more matrices of, e.g., position, orientation, and/or acceleration data as depicted in FIG. 2B. In some embodiments, multiple sensor data matrices may be used and/or combined into one or more matrices. In some embodiments, during or after capture, each sensor may transmit data, e.g., position and orientation (PnO) data, at a predetermined frequency, such as 200 Hz. For instance, data may include a position in the form of three-dimensional coordinates and rotational measures around each of three axes. In some embodiments, each sensor may measure IMU data and may transmit acceleration data, as well as PnO data.

At step 410, the VR engine plots one or more of position, rotation, and acceleration data against time as one or more graphs. In some embodiments, the VR engine may plot one or more of position, rotation, and acceleration, for one or more sensors, on a chart against another variable, e.g., time. For instance, for each unit of time, each of several sensors may produce a matrix of position, orientation, and acceleration data (e.g., as depicted in FIG. 2B) that may be plotted to produce a graph. In some embodiments, a graph may comprise many measurements (e.g., height, width, depth, pitch, roll, yaw, and acceleration in three directions) over a period of time, from a plurality of sensors (e.g., back, trunk, left and right hands, left and right arms, head, legs, knees, feet etc.).

At step 412, the VR engine generates a movement signature based on the one or more graphs. Generally, generating a movement signature comprises generating a visual or mathematical representation of data describing a movement. For instance, a movement signature may be generated based on plotting one or more of position, rotation, and acceleration, for one or more sensors, on a chart against another variable, e.g., time. Some embodiments may generate a movement signature represented by a graph with many measurements (e.g., height, width, depth, pitch, roll, yaw, and acceleration in three directions) over a period of time, from a plurality of sensors (e.g., back, trunk, left and right hands, left and right arms, head, legs, knees, feet etc.). In some embodiments, a movement signature may be generated based on collecting one or more of position, rotation, and acceleration data for a specific sensor or body part in a data structure with a corresponding time. In some embodiments, generating a movement signature may comprise performing one or more mathematical operations to one or more graphs, e.g., rounding, averaging, integrations, derivations, regression analysis, Fourier transforms, etc.

Movement signatures may be very basic representations of a movement or activity. For example, Teeth brushing signature 110 of FIG. 1A may be considered a representation of a corresponding movement signature based on changes of a hand on a vertical y-axis over time, which is not necessarily drawn to scale. Hand washing signature 120 of FIG. 1B may be considered a representation of a corresponding movement signature based on changes of a hand on a vertical y-axis over time, which is not necessarily drawn to scale. Refrigerator opening signature 130 of FIG. 1C may be considered a representation of a corresponding movement signature based on changes of a hand on a horizontal z-axis over time, which is not necessarily drawn to scale.

At step 414, the VR engine adds the movement signature to a movement library. A movement library may record movement data so that motions and activities may be used to help present movement form, as well as classify future movement. Movements and activities (e.g., ADLs) such as brushing teeth, washing hands, and opening a refrigerator door may be incorporated into a movement library. In some embodiments, a movement library may include data describing proper motions for various ADLs and activities. In some embodiments, each movement of a movement library may have a movement signature. A movement library may comprise a user interface that provides an avatar animation of an ADL, e.g., scenario 100A. In some embodiments, scenario 100A may be a portion of a VR application or activity used to help train patients.

In some embodiments, adding a movement signature to a movement library may comprise creating an activity entry for the movement signature and adding the entry to a movement library data structure. For instance, movement library 221 in data structure 200 of FIG. 2A depicts an exemplary movement library with a plurality of movements, e.g., each associated with a movement signature.

FIG. 4B depicts an illustrative flowchart of a process for extracting micromovements from a movement, in accordance with some embodiments of the disclosure. In some embodiments, micromovements may be incorporated in a movement signature and may be extracted. Micromovements may be considered smaller portions of a movement required to perform the movement. For instance, a hand-washing movement by a patient (or motion capture actor) may comprise soaping his hands, lowering his hands under a faucet, and rinsing his hands in separate steps. For instance, as depicted in FIG. 1B, hand-washing movement comprises at least three smaller movements (e.g., micromovements) including lather action 122, lowering action 124, and rinsing action 126. In some embodiments, such as scenario 100 of FIG. 1A, a teeth-brushing movement may comprise raising action 112 and brushing action 114.

In some embodiments, signatures of micromovements may be incorporated in a movement signature. In some embodiments, micromovements, such as lather action, lowering action, and rinsing action, may each have their own movement signature (e.g., within a larger movement signature). In some embodiments, micromovements may be identified within a movement signature. There are many ways to extract smaller movements and micromovements and process 450 is one example. Breaks in a movement signature may signify a change in micromovement as part of a larger movement.

Generally, process 450 of FIG. 4B includes steps for several sensors capturing movement performed by a user (e.g., position, rotation, and acceleration), receiving the sensor data from the sensors, generating a movement signature based on the sensor data, identifying breaks in the movement signature, and extracting micromovements based on the identified breaks. Some embodiments may utilize a VR engine to perform one or more parts of process 450, e.g., as part of a VR application, stored and executed by one or more of the processors and memory of a headset, server, tablet and/or other device.

At step 452, a first sensor captures a movement performed by a user. A first sensor may be one of a plurality of sensors placed on a user's body, e.g., as depicted in FIG. 7C. For instance, in a motion such as teeth brushing, as depicted in FIG. 1A, a right hand sensor may be considered a first sensor. In some embodiments, only one sensor may be used. For instance, in some cases, measuring motion of one hand may be sufficient to measure and identify a movement.

At step 454, a second sensor captures the movement performed by a user. A second sensor may be one of a plurality of sensors placed on a user's body, e.g., as depicted in FIG. 7C. For instance, in a motion such as teeth brushing, as depicted in FIG. 1A, a right arm sensor may be considered a second sensor.

At step 456, an N sensor captures the movement performed by a user. An N sensor may be one of a plurality of sensors placed on a user's body, e.g., as depicted in FIG. 7C. For instance, in a motion such as teeth brushing, as depicted in FIG. 1A, a head, back, or trunk sensor may be considered a second sensor. In some embodiments, N may any number greater than two. In some embodiments, such as sensor data depicted in FIG. 2B and sensor positions in FIG. 7C, there may be seven or more sensors, including, e.g., left hand, right hand, left arm, right arm, trunk, back, and HMD.

At step 458, the VR engine receives movement input from a plurality of sensors. Generally, each sensor may detect position and orientation (PnO) data and transmit such data to the HMD for processing. In some embodiments, each sensor may detect and transmit acceleration data e.g., based on inertial measurement units (IMUs), described in FIG. 9. In some embodiments, measured acceleration data may be used to improve accuracy of PnO data.

At step 460, the VR engine generates a movement signature based on the movement input. Generally, generating a movement signature comprises generating a visual or mathematical representation of data describing a movement. For instance, a movement signature may be a graph of sensor data over time. In some embodiments, generating a movement signature may include steps of plotting one or more portions of sensor data as graphs and generating a movement signature based on the graphs. Process 400 of FIG. 4A describes an exemplary process for generating a movement signature and adding the movement signature to a movement library. In some embodiments, a movement signature may comprise normalized values of data describing one or more movements. In some embodiments, a movement signature may comprise values in one or more matrices or other data structures. In some embodiments, a movement signature may comprise a movement data expressed as a wave. In some embodiments, a movement signature may comprise movement data expressed as a function and/or equation. In some embodiments, a movement signature may comprise VR avatar skeletal movement data based on the sensor data. In some embodiments, a movement signature may comprise VR avatar animation data and/or images.

At step 462, the VR engine identifies breaks in the movement signature. Breaks in a movement signature may signify a change in micromovement as part of a larger movement. In some embodiments, breaks may be identified by, e.g., abrupt changes in position, rotation, and/or acceleration in one or more directions. For instance, a large change in height position (e.g., y-position) may indicate a lifting or lowering micromovements. A large change in horizontal position (e.g., z-position) may indicate a pulling or pushing micromovements. In some embodiments, breaks may be identified by brief or instantaneous pauses in activity. In some embodiments, breaks may be identified by changes in patterns of motion, such as when oscillating starts or stops, or when raising or lowering starts/stops. In some embodiments, a machine learning model may be trained to identify changes between micromovements within a movement.

At step 464, the VR engine extracts a plurality of micromovements based on the identified breaks. For instance, as depicted in FIG. 1B, in the hand-washing movement, there are at least three micromovements including lather action 122, lowering action 124, and rinsing action 126. Each of those micromovements may be identified within the hand-washing movement signature. For instance, hand washing signature 120 of FIG. 1B depicts a graph of a y-position of a hand sensor, which comprises an initial oscillating portion of the curve that appears to match lather action 122, a long and steep part of the curve appears to match lowering action 124, and a latter oscillating portion of the curve appears to match rinsing motion 126.

At step 466, the VR engine outputs a first micromovement. For instance, as depicted in FIG. 1B, in the hand-washing movement, a first output micromovement may include lather action 122. In some embodiments, each micromovement may be stored in a movement library and associated with a movement or activity, along with a movement signature (or portion of a movement signature).

At step 468, the VR engine outputs a second micromovement. For instance, as depicted in FIG. 1B, in the hand-washing movement, a second output micromovement may include lowering action 124.

At step 470, the VR engine outputs an M micromovement. For instance, as depicted in FIG. 1B, in the hand-washing movement, an M output micromovement may include rinsing action 126. In some embodiments, M may be a number such as one or greater, e.g., four (4).

FIG. 5 depicts an illustrative flow diagram of a process for training a movement classifier model, in accordance with some embodiments of the disclosure. In some embodiments, classifying a movement may be accomplished with predictive modeling. For instance, a trained neural network may be used to classify provided movement data or sensor data video as one of several activities either a transition movement or not a transition movement. Generally, a training set comprising movement data comprising various movements and corresponding activity labels may be used by a neural network to classify new movement data with an activity label. In some embodiments, a multivariate time series model may be trained and used. For instance, a machine learning model similar to RocketML may be trained to classify movements based on input movement data from sensors, avatar skeletons, motion capture, etc. In some embodiments, a hierarchical model may be trained and used to classify movements.

Training models to accurately classify movements may be accomplished in many ways. Some embodiments may use supervised learning where, e.g., a training data set includes labels identifying movements by an activity label. Some embodiments may use unsupervised learning that may classify movements in training data by clustering similar data. Some embodiments may use semi-supervised learning where a portion of labeled movement data may be combined with unlabeled data during training. In some embodiments, reinforcement learning may be used. With reinforcement learning, a predictive model is trained from a series of actions by maximizing a “reward function,” via rewarding correct labeling and penalizing improper labeling. Process 500 includes data labels 512, indicating a supervised or semi-supervised learning situation. A trained model may return a movement labeled by an activity category describing the movement or may simply be labeled as similar to other movements (or not).

Process 500 depicts training movement data 510 along with data labels 512. Training data for transition movement identification may be collected by manually labeling training movements that are transition movements. In some embodiments, movement data may comprise VR sensor data, VR avatar skeletal data, movement signature data, and/or other data. Movement data without an activity classification, e.g., from a control group, may also be captured and used. In some embodiments, a capture session may be performed by a motion-capture performer (and supervised by a doctor, therapist, VR therapy application designer, etc.) performing the activity, e.g., brushing teeth, washing hands, drinking water, opening a refrigerator, showering, using a toilet, etc., to capture training data of motions for the VR system. In some circumstances, an analyst may mark captured movement data with a label of the corresponding activity, e.g., in near real time, to create the training data set. From the movement data collected, at least two groups of data may be created: training movement data 510 and test data 524.

In process 500, training movement data 510 is pre-processed using feature extraction to form training movement features 516. Pre-processing of training data is used to obtain proper data for training. In some embodiments, pre-processing may involve, for example, scaling, transforming, rotating, converting, normalizing, changing of bases, and/or translating coordinate systems in video and/or audio movement data. In some embodiments, pre-processing may involve filtering video and/or audio movement data, e.g., to eliminate video and/or audio movement noise.

After pre-processing, training movement features 516 are fed into Machine Learning Algorithm (MLA) 520 to generate an initial machine learning model, e.g., movement classifier model 540. Different types of movement classification problem spaces may require several different models. In some embodiments, MLA 520 may comprise multivariate time series classification models. In some embodiments, MLA 520 may comprise hierarchical multivariate time series classification models. In some embodiments, time series data may undergo feature engineering, such as extrapolating averages or other calculated values of sensor data, e.g., to not limit the MLA 520 to solely time-series based algorithms. In some embodiments, MLA 520 uses numbers between 0 and 1 to determine whether the provided data, e.g., training movement features 516, includes a transition movement or not. The more data that is provided, the more accurate MLA 520 will be in creating a model, e.g., movement classifier model 540.

Once MLA 520 creates movement classifier model 540, test data may be fed into the model to verify the system and evaluate how strongly model 540 behaves based on metrics such as accuracy, precision, specificity, and/or recall. In some embodiments, an additional subset of test data may be reserved for hyperparameter tuning to maximize performance of model 540. In some embodiments, test data 524 is pre-processed to become a movement feature 536 and passed to movement classifier model 540 for a classification. Movement classifier model 540 identifies an activity classification label for the input test data. In some embodiments, each iteration of test data 524 is classified and evaluated for performance based on metrics such as accuracy, precision, specificity, and/or recall. For example, if expected label 550 is not correct, false result 552 may be fed as learning data back into MLA 520. If, after test data 524 is classified and reviewed, model 540 does not perform as expected (e.g., accuracy below 75%) then additional training data may be provided until the model meets the expected criteria. In some embodiments, a poorly performing model may necessitate replacement with a higher performing algorithm. In some embodiments, a reinforcement learning method may be incorporated with test data to reward or punish MLA 520.

Once movement classifier model 540 performs at an acceptable level, new real-time data may be fed to the model, and determinations of whether the data may be classified as a particular activity with confidence. For instance, patient motion may be input and classified as a particular activity or movement. In some embodiments, such as process 500, new movement data 530 may be pre-processed as a movement feature 536 and passed to movement classifier model 540 for a classification as an activity. Movement classifier model 540 may evaluate movement feature 536 and present classification label 550 based on the input data. If a classification label 550 for new movement data 530 can be verified outside the system, model 540 may be further updated with feedback and reinforcement for further accuracy.

In some embodiments, user data (e.g., a patient profile) may be input with movement data. For instance, a patient's height, weight, sex, age, body mass, and impairment and/or health history may affect a movement. Likewise, a movement classifier model may take into account patient qualities that may help the model better predict and classify movement. For instance, data coming from patients with movement disorders may be used to identify a motion by another patient suffering from a similar movement disorder. In some embodiments, a movement classifier model may be used to help identify movement similar to patients suffering from particular disorders and suggest a diagnosis.

FIG. 6 depicts an illustrative flowchart of a process for selecting a VR therapy activity appropriate for practicing a movement, in accordance with some embodiments of the disclosure. There are many ways to identify appropriate VR activities and sub-activities for treating a patient and process 600 is one example. Generally, process 600 of FIG. 6 includes steps for comparing micromovements from goal ADLs to the micromovements exercised in available VR activities, determining if the micromovements match, e.g., above a threshold, and providing a subset of activities matching the micromovements of a patient's ADL goals.

Some embodiments may utilize a VR engine to perform one or more parts of process 600, e.g., as part of a VR application, stored and executed by one or more of the processors and memory of a headset, server, tablet and/or other device. For instance, VR engine may be incorporated in one or more of head-mounted display 201 and clinician tablet 210 of FIGS. 7A-D and/or the systems of FIGS. 9-10. A VR engine may run on a component of a tablet, HMD, server, display, television, set-top box, computer, smartphone, or other device.

At step 602, the VR engine accesses the micromovements used in Activity 1. For instance, with steps 602, 604, and 606, there may be a library of VR activities and each VR activity is associated with at least one movement. Each movement may be broken down into one or more micromovements. In some embodiments, a movement library comprising various activities may be used to store activities, movements, and micromovements. For instance, movement library 221 in data structure 200 of FIG. 2A depicts an exemplary movement library with a plurality of movements, e.g., each associated with a movement signature available for user participation. Micromovements may be determined in several ways. For instance, process 450 of FIG. 4B describes a process for outputting micromovements.

By way of a nonlimiting example, Activity 1 may be considered an activity titled “Feed the Squirrels” as depicted as VR activity 172 in FIG. 1D. A list of micromovements associated with Activity 1, e.g., lifting an item, may be stored with application and activity data in a database or movement library. An exemplary data structure for storing movement library information, including activities, movements, and micromovements, is depicted in scenario 200 of FIG. 2A. In some embodiments, Activity 1 (e.g., “Feed the Squirrels”) may direct the user to complete different micromovements, e.g., lifting their hand, lowering their hand, choosing a specific spoon or food type, grabbing food or the spoon, and other micromovements. In some embodiments, micromovements within the activities may be prioritized or weighted based on prevalence within the activity. For instance, an activity's focus on lifting may be a tier 1 micromovement (e.g., weighted at 100%) while reaching for an item may be a tier 2 or lower micromovement (e.g., weighted at 80%) because it is requested less frequently. Different activities may have different scores or weights for various micromovements.

At step 604, the VR engine accesses a list of micromovements used in Activity 2. For example, Activity 2 may be considered a virtual therapy activity titled “Start the Campfire,” depicted as VR activity 174 of FIG. 1D. In Activity 2, “Start the Campfire,” may include a micromovement of a user rubbing their hands together like rubbing a stick to start a fire, e.g., to help practice elements lathering and rinsing with hand washing, among other micromovements and ADLs.

At step 606, the VR engine receives a list of micromovements in Activity Q. In some embodiments, Activity Q may represent the last of Q activities potentially available in the movement library. Some embodiments may feature a several available activities, while some other embodiments may include dozens or more VR applications and/or activities. For instance, Activity Q may be considered an activity titled “Find the Woodland Friends,” e.g., depicted as VR activity 178 of FIG. 1D. Activity Q may include, e.g., micromovements of opening doors, among others, to exercise micromovements that may be beneficial to, e.g., opening a refrigerator, a cupboard, a medicine cabinet, or other actions.

At step 608, the VR engine receives an input associated with a first movement related to a specific activity, such as an ADL. This first movement may be a problematic movement for a patient or a very essential motion that a particular patient needs to practice, e.g., with fun, challenging, immersive therapy. In some embodiments, this may be a simple selection on a user interface of an activity a patient needs to improve to gain more independence. In some embodiments, the input may be performance of a movement by a patient that falls short of a proper motion. In some embodiments, a patient profile may be used to identify and select a first movement. For instance, a patient may be participating in VR therapy and her profile is prepared for access by the VR engine. In scenario 140, patient profile 152 for “Jane Doe” is received. A patient, for example, may need to work on the ADLs of teeth brushing, hand washing, and refrigerator opening. These activities may then be added to their profile 152 and prepared for access by the VR engine, as demonstrated in FIG. 1D. Patient profiles may be stored in a secure database, e.g., as depicted in FIG. 10. Like in FIG. 1D, the received patient profile may be considered to list ADLs, such as teeth brushing and hand washing, and hold the associated micromovements in their data structure.

In some embodiments, the first activity, e.g., from the input, may be divided (or previously divided) into a plurality of micromovements. An exemplary process for extracting micromovements may be found in process 300 of FIG. 3A. In some embodiments, ADLs and/or micromovements in a profile may be prioritized or weighted based on the patient's needs. For instance, a patient's focus on lifting may be a tier 1 micromovement (e.g., weighted at 100%) while reaching for an item may be a tier 2 or lower tiered micromovement (e.g., weighted at 80%). Such weighting may be set by a doctor or therapist or be set as a default, e.g., according to one or more particular ailments or impairments. Different movements required by a patient's day-to-day activities may have different scores or weights for various micromovements. For instance, someone living in assisted living may need to focus more on basic ADLs than someone living a more independent lifestyle. Likewise, a young person rehabilitating a torn shoulder ligament may need to focus more on activities with upward reaching than, e.g., someone recovering from a stroke who may need to exercise movements in basic ADLs like drinking and self-feeding. In some embodiments, a patient profile may be received prior to or when a patient logs into the system and/or begins a therapy session.

At step 610, the VR engine compares the micromovements identified in the patient profile to each activity's list of included micromovements. In some embodiments, micromovements from each activity's list and the patient profile may be compared. In some embodiments, matches between a patient profile and an activity's movement list are identified and counted. In some embodiments, matches from each activity's list may be prioritized or weighted based on prevalence within the activity or in the patient profile. For instance, matches may be identified and weighted based on a tier of each micromovement (e.g., prioritization). A match of an activity prioritizing brushing teeth for a patient with minimal ability to do so may be weighted (e.g., 125%) more than a match with an activity focusing on working memory when the patient has only minor memory issues (e.g., 50%).

Comparisons may be performed in several ways. In some embodiments, micromovements from each activity's list and the patient profile may be correlated and a match score (e.g., 1-100) for the activity calculated. For instance, each micromovement included in an ADL in a patient profile and each VR activity may be given a numeric identifier and a weight value. In some embodiments, numeric identifiers and each corresponding weight value for a profile or VR activity may form matrices and the matrices are correlated. In some embodiments, numeric identifiers and each corresponding weight value for a profile or VR activity may be charted as coordinates and use linear regression to compare. In some embodiments, an index of every micromovement in all of the applications may be used, wherein each micromovement is associated with one or more applications and/or activities that may include the micromovement or ADL as a whole.

In some embodiments, a comparison may be made by a trained model using, e.g., a neural network. For instance, a model may be trained to accept a patient profile as input and identify one or more VR activities suitable for the patient profile. Such a model may be trained by doctors and/or therapists who prove training data of profiles and identify which VR activities may be appropriate for use. Then, using a feedback loop, the model may be further trained with test patient profiles by, e.g., rewarding the neural network for correct predictions of suitable VR activities and retraining with incorrect predictions. In some embodiments, a comparison may use a combination of a trained model and comparative analysis.

At step 620, for each activity, the VR engine determines whether the activity's micromovements match the micromovements identified in the patient's profile, e.g., above a predetermined threshold. For instance, if the counted matches between an activity and the patient profile meet or exceed a threshold (e.g., five matches), the activity may be further analyzed. In some embodiments, e.g., when a weighting or correlation is used, the threshold may be a score from 1-100, such as 75, and the activity match score must meet or exceed the match score threshold. In some embodiments, the threshold may be based on the number of micromovements in a patient profile, e.g., the threshold may be two-thirds (e.g., 66%) of the total number of micromovements in a profile. In some embodiments, the match threshold may be one match, e.g., in situation where patients have only one or a couple micromovements or ADLs.

If the VR engine determines an analyzed activity's micromovements do not match the micromovements identified in the patient's profile above a threshold, then, at step 622, the VR engine does not add activity to a subset of activities for further analysis. For instance, if the threshold is five matches and the activity only has two matches, the activity is discarded for now. In some embodiments, if the threshold is a match score of 75 (e.g., on a 0-99 scale) and the activity only has a match score of 40, the activity is discarded for now. In some embodiments, if all the activities are evaluated for matches and none meet the predetermined threshold, a second (lower) predetermined threshold may be used (e.g., half or two-thirds of the first threshold).

If the VR engine determines an activity's micromovements do match the micromovements identified in the patient's profile above a threshold, then, at step 624, the VR engine adds the matching activity to a subset of activities. For instance, if the threshold is four matches and the activity has six matches, the activity is added to the subset for further review. In some embodiments, if the threshold is a match score of 85 (on a 0-99 scale) and the activity has a match score of 92, the activity is added to the subset for further review.

At step 626, the VR engine accesses more information for each activity of subset of activities. For instance, additional information for an activity may comprise calendar data of when the activity was last accessed, compatibility data, activity version and update data, average activity duration data, activity performance data, and other data. For instance, some additional activity data may indicate recent participation in an activity and/or recent success/struggles with the activity. In some embodiments, additional information may include recommendation/weighting by a doctor or therapist indicating a preference to use (or not use) a particular motion required by one or more activities. In some embodiments, an activity may be eliminated from the subset if, e.g., a conflict arises based on additional activity data. In some embodiments, a warning of a potential conflict may be provided.

At step 628, the VR engine provides one or more activities from the subset of activities. For instance, scenario 140 of FIG. 1D depicts a menu for a VR therapy platform suggesting VR activities 172, 174, 176, and 178 based on an identified patient profile 172. In scenario 140, the matches between profile 152 and each of activities 172, 174, 176, and 178 are apparent. Patient profile 152 for “Jane Doe” indicates difficulty or discomfort with, e.g., teeth brushing, hand washing, refrigerator opening. For instance, Activity 1 from step 602, “Feed the Squirrels” (e.g., VR activity 172 of FIG. 1D), may include movements of lifting an object up and down to feed squirrels and/or choosing what to feed the squirrels, among other movements. Activity 2 from step 604, “Start the Campfire” (VR activity 174 of FIG. 1D), may include rubbing two sticks together, among other movements. Activity 1 may be ranked higher than Activity 2 because, e.g., there are more matching micromovements. In some embodiments, Activity 1 may be ranked ahead of Activity 2 because Activity 2 requires movement that may adversely impact the patient, in accordance with data in the patient profile. Activity Q from step 606, “Find the Woodland Friends,” may include opening doors, among other movements. While Activity Q from step 606, “Find the Woodland Friends,” may have some matches with the Jane Doe profile, the matches do not merit ranking as high as, e.g., Activity 1, “Feed the Squirrels,” or may rank above, e.g., Activity 2, “Start the Campfire.”

As further disclosed herein is an illustrative medical device system including a virtual reality system to enable therapy for a patient. Such a VR medical device system may include a headset, sensors, a therapist tablet, among other hardware to enable exercises and activities to train (or re-train) a patient's body movements.

As described herein, VR systems capable for use in physical therapy may be tailored to be durable, portable and allow for quick and consistent setup. In some embodiments, a virtual reality system for therapy may be a modified commercial VR system using, e.g., a headset and several body sensors configured for wireless communication. A VR system capable of use for therapy may need to collect patient movement data. In some embodiments, sensors, placed on the patient's body, can translate patient body movement to the VR system for animation of a VR avatar. Sensor data may also be used to measure patient movement and determine motion for patient body parts.

FIG. 7A is a diagram of an illustrative system, in accordance with some embodiments of the disclosure. A VR system may include a clinician tablet 210, head-mounted display 201 (HMD or headset), small sensors 202, and large sensor 202B. Large sensor 202B may comprise transmitters, in some embodiments, and be referred to as wireless transmitter module 202B. Some embodiments may include sensor chargers, router, router battery, headset controller, power cords, USB cables, and other VR system equipment.

Clinician tablet 210 may be configured to use a touch screen, a power/lock button that turns the component on or off, and a charger/accessory port, e.g., USB-C. For instance, pressing the power button on clinician tablet 210 may power on the tablet or restart the tablet. Once clinician tablet 210 is powered on, a therapist or supervisor may access a user interface and be able to log in; add or select a patient; initialize and sync sensors; select, start, modify, or end a therapy session; view data; and/or log out.

Headset 201 may comprise a power button that turns the component on or off, as well as a charger/accessory port, e.g., USB-C. Headset 201 may also provide visual feedback of virtual reality applications in concert with the clinician tablet and the small and large sensors.

Charging headset 201 may be performed by plugging a headset power cord into the storage dock or an outlet. To turn on headset 201 or restart headset 201, the power button may be pressed. A power button may be on top of the headset. Some embodiments may include a headset controller used to access system settings. For instance, a headset controller may be used only in certain troubleshooting and administrative tasks and not necessarily during patient therapy. Buttons on the controller may be used to control power, connect to headset 201, access settings, or control volume.

The large sensor 202B and small sensors 202 are equipped with mechanical and electrical components that measure position and orientation in physical space and then translate that information to construct a virtual environment. Sensors 202 are turned off and charged when placed in the charging station. Sensors 202 turn on and attempt to sync when removed from the charging station. The sensor charger acts as a dock to store and charge the sensors. In some embodiments, sensors may be placed in sensor bands on a patient. Sensor bands 205, as depicted in FIGS. 7B-C, are typically required for use and are provided separately for each patient for hygienic purposes. In some embodiments, sensors may be miniaturized and may be placed, mounted, fastened, or pasted directly onto a user.

As shown in illustrative FIG. 7A, various systems disclosed herein consist of a set of position and orientation sensors that are worn by a VR participant, e.g., a therapy patient. These sensors communicate with HMD 201, which immerses the patient in a VR experience. An HMD suitable for VR often comprises one or more displays to enable stereoscopic three-dimensional (3D) images. Such internal displays are typically high-resolution (e.g., 2880×1600 or better) and offer high refresh rate (e.g., 75 Hz). The displays are configured to present 3D images to the patient. VR headsets typically include speakers and microphones for deeper immersion.

HMD 201 is a piece central to immersing a patient in a virtual world in terms of presentation and movement. A headset may allow, for instance, a wide field of view (e.g., 110°) and tracking along six degrees of freedom. HMD 201 may include cameras, accelerometers, gyroscopes, and proximity sensors. VR headsets typically include a processor, usually in the form of a system on a chip (SoC), and memory. In some embodiments, headsets may also use, for example, additional cameras as safety features to help users avoid real-world obstacles. HMD 201 may comprise more than one connectivity option in order to communicate with the therapist's tablet. For instance, an HMD 201 may use an SoC that features WiFi and Bluetooth connectivity, in addition to an available USB connection (e.g., USB Type-C). The USB-C connection may also be used to charge the built-in rechargeable battery for the headset.

A supervisor, such as a health care provider or therapist, may use a tablet, e.g., tablet 210 depicted in FIG. 7A, to control the patient's experience. In some embodiments, tablet 210 runs an application and communicates with a router to cloud software configured to authenticate users and store information. Tablet 210 may communicate with HMD 201 in order to initiate HMD applications, collect relayed sensor data, and update records on the cloud servers. Tablet 210 may be stored in the portable container and plugged in to charge, e.g., via a USB plug.

In some embodiments, such as depicted in FIGS. 7B-C, sensors 202 are placed on the body in particular places to measure body movement and relay the measurements for translation and animation of a VR avatar. Sensors 202 may be strapped to a body via bands 205. In some embodiments, each patient may have her own set of bands 205 to minimize hygiene issues.

A wireless transmitter module (WTM) 202B may be worn on a sensor band 205B that is laid over the patient's shoulders. WTM 202B sits between the patient's shoulder blades on their back. Wireless sensor modules 202 (e.g., sensors or WSMs) are worn just above each elbow, strapped to the back of each hand, and on a pelvis band that positions a sensor adjacent to the patient's sacrum on their back. In some embodiments, each WSM communicates its position and orientation in real-time with an HMD Accessory located on the HMD. Each sensor 202 may learn its relative position and orientation to the WTM, e.g., via calibration.

The HMD accessory may include a sensor 202A that may allow it to learn its position relative to WTM 202B, which then allows the HMD to know where in physical space all the WSMs and WTM are located. In some embodiments, each sensor 202 communicates independently with the HMD accessory which then transmits its data to HMD 201, e.g., via a USB-C connection. In some embodiments, each sensor 202 communicates its position and orientation in real-time with WTM 202B, which is in wireless communication with HMD 201. In some embodiments HMD 201 may be connected to input supplying other data such as biometric feedback data. For instance, in some cases, the VR system may include heart rate monitors, electrical signal monitors, e.g., electrocardiogram (EKG), eye movement tracking, brain monitoring with Electroencephalogram (EEG), pulse oximeter monitors, temperature sensors, blood pressure monitors, respiratory monitors, light sensors, cameras, sensors, and other biometric devices. Biometric feedback, along with other performance data, can indicate more subtle changes to the patient's body or physiology as well as mental state, e.g., when a patient is stressed, comfortable, distracted, tired, over-worked, under-worked, over-stimulated, confused, overwhelmed, excited, engaged, disengaged, and more. In some embodiments, such devices measuring biometric feedback may be connected to the HMD and/or the supervisor tablet via USB, Bluetooth, Wi-Fi, radio frequency, and other mechanisms of networking and communication.

A VR environment rendering engine on HMD 201 (sometimes referred to herein as a “VR application”), such as the Unreal Engine™, uses the position and orientation data to create an avatar that mimics the patient's movement.

A patient or player may “become” their avatar when they log in to a virtual reality activity. When the player moves their body, they see their avatar move accordingly. Sensors in the headset may allow the patient to move the avatar's head, e.g., even before body sensors are placed on the patient. A system that achieves consistent high-quality tracking facilitates the patient's movements to be accurately mapped onto an avatar.

Sensors 202 may be placed on the body, e.g., of a patient by a therapist, in particular locations to sense and/or translate body movements. The system can use measurements of position and orientation of sensors placed in key places to determine movement of body parts in the real world and translate such movement to the virtual world. In some embodiments, a VR system may collect performance data for therapeutic analysis of a patient's movements and range of motion.

In some embodiments, systems and methods of the present disclosure may use electromagnetic tracking, optical tracking, infrared tracking, accelerometers, magnetometers, gyroscopes, myoelectric tracking, other tracking techniques, or a combination of one or more of such tracking methods. The tracking systems may be parts of a computing system as disclosed herein. The tracking tools may exist on one or more circuit boards within the VR system (see FIG. 9) where they may monitor one or more users to perform one or more functions such as capturing, analyzing, and/or tracking a subject's movement. In some cases, a VR system may utilize more than one tracking method to improve reliability, accuracy, and precision.

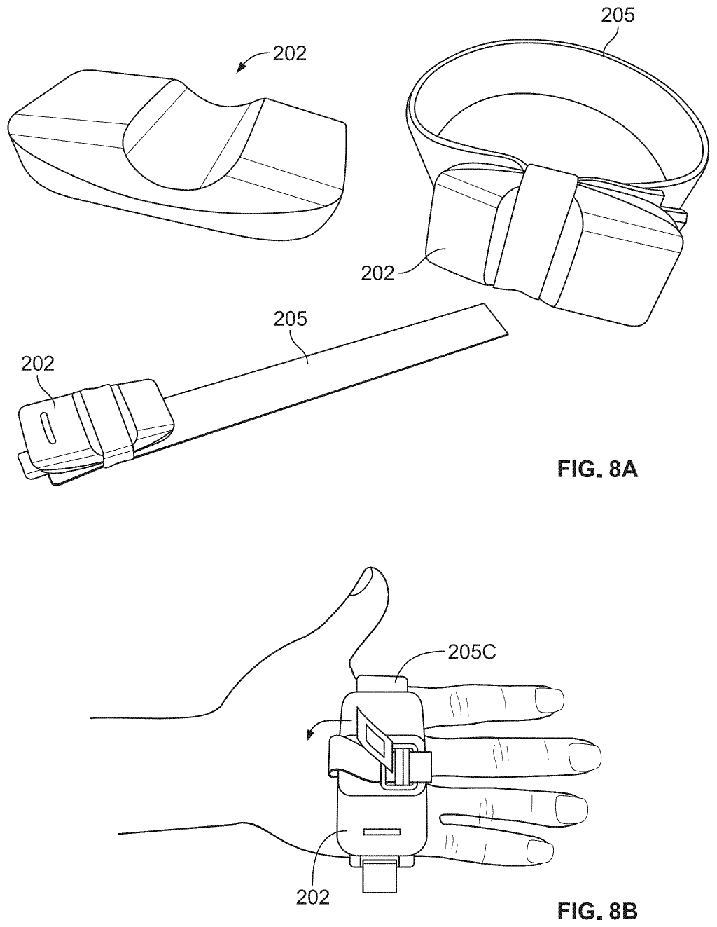

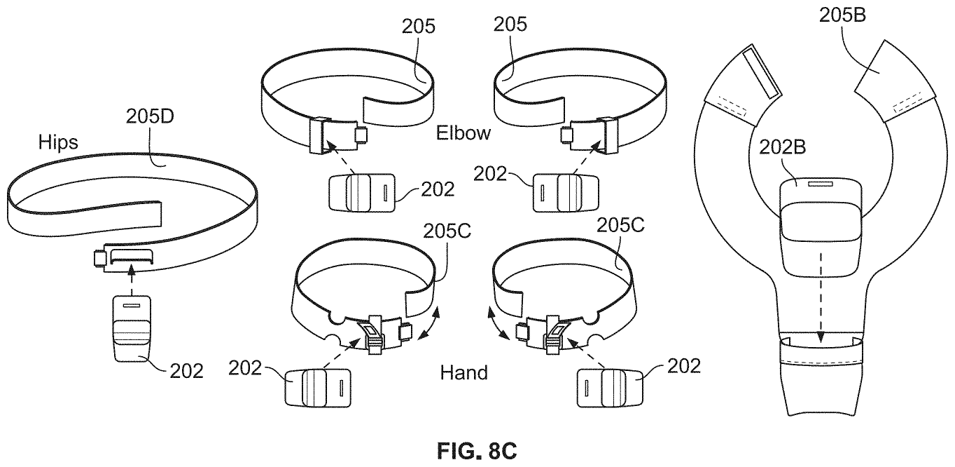

FIGS. 8A-C illustrate examples of wearable sensors 202 and bands 205. In some embodiments, bands 205 may include elastic loops to hold the sensors. In some embodiments, bands 205 may include additional loops, buckles and/or Velcro straps to hold the sensors. For instance, bands 205 for hands may require extra secureness as a patient's hands may be moved at a greater speed and could throw or project a sensor in the air if it is not securely fastened. FIG. 8B illustrates an exemplary embodiment with a slide buckle.

Sensors 202 may be attached to body parts via band 205. In some embodiments, a therapist attaches sensors 202 to proper areas of a patient's body. For example, a patient may not be physically able to attach band 205 to herself. In some embodiments, each patient may have her own set of bands 205 to minimize hygiene issues. In some embodiments, a therapist may bring a portable case to a patient's room or home for therapy. The sensors may include contact ports for charging each sensor's battery while storing and transporting in the container, such as the container depicted in FIG. 7A.

As illustrated in FIG. 8C, sensors 202 are placed in bands 205 prior to placement on a patient. In some embodiments, sensors 202 may be placed onto bands 205 by sliding them into the elasticized loops. The large sensor, WTM 202B, is placed into a pocket of shoulder band 205B. Sensors 202 may be placed above the elbows, on the back of the hands, and at the lower back (sacrum). In some embodiments, sensors may be used at the knees and/or ankles. Sensors 202 may be placed, e.g., by a therapist, on a patient while the patient is sitting on a bench (or chair) with his hands on his knees. Sensor band 205D to be used as a hip sensor 202 has a sufficient length to encircle a patient's waist.

Once sensors 202 are placed in bands 205, each band may be placed on a body part, e.g., according to FIG. 7C. In some embodiments, shoulder band 205B may require connection of a hook and loop fastener. An elbow band 205 holding a sensor 202 should sit behind the patient's elbow. In some embodiments, hand sensor bands 205C may have one or more buckles to, e.g., fasten sensors 202 more securely, as depicted in FIG. 8B.

Each of sensors 202 may be placed at any of the suitable locations, e.g., as depicted in FIG. 7C. After sensors 202 have been placed on the body, they may be assigned or calibrated for each corresponding body part.

Generally, sensor assignment may be based on the position of each sensor 202. Sometimes, such as cases where patients have varying height discrepancies, assigning a sensor merely based on height is not practical. In some embodiments, sensor assignment may be based on relative position to, e.g., wireless transmitter module 202B.

FIG. 9 depicts an illustrative arrangement for various elements of a system, e.g., an HMD and sensors of FIGS. 7A-D. The arrangement includes one or more printed circuit boards (PCBs). In general terms, the elements of this arrangement track, model, and display a visual representation of the participant (e.g., a patient avatar) in the VR world by running software including the aforementioned VR application of HMD 201.

The arrangement shown in FIG. 9 includes one or more sensors 902, processors 960, graphic processing units (GPUs) 920, video encoder/video codec 940, sound cards 946, transmitter modules 910, network interfaces 980, and light emitting diodes (LEDs) 969. These components may be housed on a local computing system or may be remote components in wired or wireless connection with a local computing system (e.g., a remote server, a cloud, a mobile device, a connected device, etc.). Connections between components may be facilitated by one or more buses, such as bus 914, bus 934, bus 948, bus 984, and bus 964 (e.g., peripheral component interconnects (PCI) bus, PCI-Express bus, or universal serial bus (USB)). With such buses, the computing environment may be capable of integrating numerous components, numerous PCBs, and/or numerous remote computing systems.

One or more system management controllers, such as system management controller 912 or system management controller 932, may provide data transmission management functions between the buses and the components they integrate. For instance, system management controller 912 provides data transmission management functions between bus 914 and sensors 902. System management controller 932 provides data transmission management functions between bus 934 and GPU 920. Such management controllers may facilitate the arrangements orchestration of these components that may each utilize separate instructions within defined time frames to execute applications. Network interface 980 may include an ethernet connection or a component that forms a wireless connection, e.g., 802.11b, g, a, or n connection (WiFi), to a local area network (LAN) 987, wide area network (WAN) 983, intranet 985, or internet 981. Network controller 982 provides data transmission management functions between bus 984 and network interface 980.

Processor(s) 960 and GPU 920 may execute a number of instructions, such as machine-readable instructions. The instructions may include instructions for receiving, storing, processing, and transmitting tracking data from various sources, such as electromagnetic (EM) sensors 903, optical sensors 904, infrared (IR) sensors 907, inertial measurement units (IMUs) sensors 905, and/or myoelectric sensors 906. The tracking data may be communicated to processor(s) 960 by either a wired or wireless communication link, e.g., transmitter 910. Upon receiving tracking data, processor(s) 960 may execute an instruction to permanently or temporarily store the tracking data in memory 962 such as, e.g., random access memory (RAM), read only memory (ROM), cache, flash memory, hard disk, or other suitable storage component. Memory may be a separate component, such as memory 968, in communication with processor(s) 960 or may be integrated into processor(s) 960, such as memory 962, as depicted.

Processor(s) 960 may also execute instructions for constructing an instance of virtual space. The instance may be hosted on an external server and may persist and undergo changes even when a participant is not logged in to said instance. In some embodiments, the instance may be participant-specific, and the data required to construct it may be stored locally. In such an embodiment, new instance data may be distributed as updates that users download from an external source into local memory. In some exemplary embodiments, the instance of virtual space may include a virtual volume of space, a virtual topography (e.g., ground, mountains, lakes), virtual objects, and virtual characters (e.g., non-player characters “NPCs”). The instance may be constructed and/or rendered in 2D or 3D. The rendering may offer the viewer a first-person or third-person perspective. A first-person perspective may include displaying the virtual world from the eyes of the avatar and allowing the patient to view body movements from the avatar's perspective. A third-person perspective may include displaying the virtual world from, for example, behind the avatar to allow someone to view body movements from a different perspective. The instance may include properties of physics, such as gravity, magnetism, mass, force, velocity, and acceleration, which cause the virtual objects in the virtual space to behave in a manner at least visually similar to the behaviors of real objects in real space.

Processor(s) 960 may execute a program (e.g., the Unreal Engine or VR applications discussed above) for analyzing and modeling tracking data. For instance, processor(s) 960 may execute a program that analyzes the tracking data it receives according to algorithms described above, along with other related pertinent mathematical formulas. Such a program may incorporate a graphics processing unit (GPU) 920 that is capable of translating tracking data into 3D models. GPU 920 may utilize shader engine 928, vertex animation 924, and linear blend skinning algorithms. In some instances, processor(s) 960 or a CPU may at least partially assist the GPU in making such calculations. This allows GPU 920 to dedicate more resources to the task of converting 3D scene data to the projected render buffer. GPU 920 may refine the 3D model by using one or more algorithms, such as an algorithm learned on biomechanical movements, a cascading algorithm that converges on a solution by parsing and incrementally considering several sources of tracking data, an inverse kinematics (IK) engine 930, a proportionality algorithm, and other algorithms related to data processing and animation techniques. After GPU 920 constructs a suitable 3D model, processor(s) 960 executes a program to transmit data for the 3D model to another component of the computing environment (or to a peripheral component in communication with the computing environment) that is capable of displaying the model, such as display 950.

In some embodiments, GPU 920 transfers the 3D model to a video encoder or a video codec 940 via a bus, which then transfers information representative of the 3D model to a suitable display 950. The 3D model may be representative of a virtual entity that can be displayed in an instance of virtual space, e.g., an avatar. The virtual entity is capable of interacting with the virtual topography, virtual objects, and virtual characters within virtual space. The virtual entity is controlled by a user's movements, as interpreted by sensors 902 communicating with the system. Display 950 may display a Patient View. The patient's real-world movements are reflected by the avatar in the virtual world. The virtual world may be viewed in the headset in 3D and monitored on the tablet in two dimensions. In some embodiments, the VR world is an activity that provides feedback and rewards based on the patient's ability to complete activities. Data from the in-world avatar is transmitted from the HMD to the tablet to the cloud, where it is stored for later analysis. An illustrative architectural diagram of such elements in accordance with some embodiments is depicted in FIG. 10.

A VR system may also comprise display 970, which is connected to the computing environment via transmitter 972. Display 970 may be a component of a clinician tablet. For instance, a supervisor or operator, such as a therapist, may securely log in to a clinician tablet, coupled to the system, to observe and direct the patient to participate in various activities and adjust the parameters of the activities to best suit the patient's ability level. Display 970 may depict a view of the avatar and/or replicate the view of the HMD.

In some embodiments, HMD 201 may be the same as or similar to HMD 1010 in FIG. 10. In some embodiments, HMD 1010 runs a version of Android that is provided by HTC (e.g., a headset manufacturer) and the VR application is an Unreal application, e.g., Unreal Application 1016, encoded in an Android package (.apk). The .apk comprises a set of custom plugins: WVR, WaveVR, SixenseCore, SixenseLib, and MVICore. The WVR and WaveVR plugins allow the Unreal application to communicate with the VR headset's functionality. The SixenseCore, SixenseLib, and MVICore plugins allow Unreal Application 1016 to communicate with the HMD accessory and sensors that communicate with the HMD via USB-C. The Unreal Application comprises code that records the position and orientation (PnO) data of the hardware sensors and translates that data into a patient avatar, which mimics the patient's motion within the VR world. An avatar can be used, for example, to infer and measure the patient's real-world range of motion. The Unreal application of the HMD includes an avatar solver as described, for example, below.

The clinician operator device, clinician tablet 1020, runs a native application (e.g., Android application 1025) that allows an operator such as a therapist to control a patient's experience. Cloud server 1050 includes a combination of software that manages authentication, data storage and retrieval, and hosts the user interface, which runs on the tablet. This can be accessed by tablet 1020. Tablet 1020 has several modules.

As depicted in FIG. 10, the first part of tablet software is a mobile device management (MDM) 1024 layer, configured to control what software runs on the tablet, enable/disable the software remotely, and remotely upgrade the tablet applications.

The second part is an application, e.g., Android Application 1025, configured to allow an operator to control the software of HMD 1010. In some embodiments, the application may be a native application. A native application, in turn, may comprise two parts, e.g., (1) socket host 1026 configured to receive native socket communications from the HMD and translate that content into web sockets, e.g., web sockets 1027, that a web browser can easily interpret; and (2) a web browser 1028, which is what the operator sees on the tablet screen. The web browser may receive data from the HMD via the socket host 1026, which translates the HMD's native socket communication 1018 into web sockets 1027, and it may receive UI/UX information from a file server 1052 in cloud 1050. Tablet 1020 comprises web browser 1028, which may incorporate a real-time 3D engine, such as Babylon.js, using a JavaScript library for displaying 3D graphics in web browser 1028 via HTMLS. For instance, a real-time 3D engine, such as Babylon.js, may render 3D graphics, e.g., in web browser 1028 on clinician tablet 1020, based on received skeletal data from an avatar solver in the Unreal Engine 1016 stored and executed on HMD 1010. In some embodiments, rather than Android Application 1026, there may be a web application or other software to communicate with file server 1052 in cloud 1050. In some instances, an application of Tablet 1020 may use, e.g., Web Real-Time Communication (WebRTC) to facilitate peer-to-peer communication without plugins, native apps, and/or web sockets.

The cloud software, e.g., cloud 1050, has several different, interconnected parts configured to communicate with the tablet software: authorization and API server 1062, GraphQL server 1064, and file server (static web host) 1052.

In some embodiments, authorization and API server 1062 may be used as a gatekeeper. For example, when an operator attempts to log in to the system, the tablet communicates with the authorization server. This server ensures that interactions (e.g., queries, updates, etc.) are authorized based on session variables such as operator's role, the health care organization, and the current patient. This server, or group of servers, communicates with several parts of the system: (a) a key value store 1054, which is a clustered session cache that stores and allows quick retrieval of session variables; (b) a GraphQL server 1064, as discussed below, which is used to access the back-end database in order to populate the key value store, and also for some calls to the application programming interface (API); (c) an identity server 1056 for handling the user login process; and (d) a secrets manager 1058 for injecting service passwords (relational database, identity database, identity server, key value store) into the environment in lieu of hard coding.

When the tablet requests data, it will communicate with the GraphQL server 1064, which will, in turn, communicate with several parts: (1) the authorization and API server 1062; (2) the secrets manager 1058, and (3) a relational database 1053 storing data for the system. Data stored by the relational database 1053 may include, for instance, profile data, session data, application data, activity performance data, and motion data.

In some embodiments, profile data may include information used to identify the patient, such as a name or an alias. Session data may comprise information about the patient's previous sessions, as well as, for example, a “free text” field into which the therapist can input unrestricted text, and a log 1055 of the patient's previous activity. Logs 1055 are typically used for session data and may include, for example, total activity time, e.g., how long the patient was actively engaged with individual activities; activity summary, e.g., a list of which activities the patient performed, and how long they engaged with each on; and settings and results for each activity. Activity performance data may incorporate information about the patient's progression through the activity content of the VR world. Motion data may include specific range-of-motion (ROM) data that may be saved about the patient's movement over the course of each activity and session, so that therapists can compare session data to previous sessions' data.

In some embodiments, file server 1052 may serve the tablet software's website as a static web host.

In some embodiments, the activities and exercises may include gazing activities that require the player to turn and look. A gaze activity may be presented as a hide-and-seek activity, a follow-and-seek exercise, or a gaze and trigger activity. The activities may include sun rising activities that require the player to raise his or her arms. The activities may include hot air balloon exercise s that require the player to lean and bend. The activities may include bird placing activities that require the player to reach and place. The exercises may include a soccer-like activity that requires a player to block and/or dodge projectiles. These activities may be presented as sandbox activities, with no clear win condition or end point. Some of these may be free-play environments presented as an endless interactive lobby. Sandbox versions of the activities may be typically used to introduce the player to the activity mechanics, and it allows them to explore the specific exercise's unique perspective of the virtual reality environment. Additionally, the sandbox activities may allow a therapist to use objects to augment and customize therapy, such as with resistance bands, weights, and the like. After the player has learned how the exercise mechanics works, they can be loaded into a version of the activity with a clear objective. In these versions of the activity, the player's movements may be tracked and recorded. After completing the prescribed number of repetitions (reps) of the therapeutic exercise (a number that is adjustable), the activity may come to an end and the player may be rewarded for completing it. In some embodiments, activities and exercises may be dynamically adjusted during the activity to optimize patient engagement and/or therapeutic benefits.

The transition from activity to activity may be seamless. Several transition options may be employed. The screen may simply fade to black, and slowly reload through a fade from black. A score board or a preview of the next exercise may be used to distract the player during transition. A slow and progressive transition ensures that the patient is not startled by a sudden change of their entire visual environment. This slow progression may limit any disorientation that might occurs from a total, quick change in scenery while in VR.

At the end of an activity or exercise session, the player may be granted a particular view of the VR environment, such as a birds-eye view of the world or area. From this height, players may be offered a view of an ever-changing village. Such changes in the village are a direct response to the player's exercise progression, and therefore offer a visual indication of progression. These changes will continue as the player progresses through the activities to provide long-term feedback visual cues. Likewise, such views of the village may provide the best visual indicia of progress for sharing with family members or on social media. Positive feedback from family and friends is especially important when rehab progress is limited. These images will help illustrate how hard the player has been working and they will provide an objective measure of progress when, perhaps, physically the player feels little, if any, progress. Such features may enhance the positivity of the therapy experience and helps fulfill the VR activities' overall goals to be as positive as possible while to encouraging continued participation and enthusiasm.

While the foregoing discussion describes exemplary embodiments of the present invention, one skilled in the art will recognize from such discussion, the accompanying drawings, and the claims, that various modifications can be made without departing from the spirit and scope of the invention. Therefore, the illustrations and examples herein should be construed in an illustrative, and not a restrictive sense. The scope and spirit of the invention should be measured solely by reference to the claims that follow.

1. A method of classifying a movement performed in a virtual reality system, the method comprising:
receiving input from a plurality of sensors;
generating a movement signature based on the input from the plurality of sensors;
determining a movement classification based on the movement signature; and
providing the movement classification.

2. The method of claim 1, wherein generating the movement signature based on the input from the plurality of sensors comprises charting time against at least one of the following from each of the plurality of sensors: position data, rotation data, and acceleration data.

3. The method of claim 1, wherein determining the movement classification comprises using a trained machine learning model to generate data indicative of a classification of the movement signature based on a plurality of stored movement signatures.

4. The method of claim 3, wherein the trained machine learning model is trained to receive the movement signature as input and output at least one movement classification describing the movement signature.

5. The method of claim 3, wherein the trained machine learning model generates data indicative of the classification of the movement signature further based on at least one of the following criteria associated with a user of the virtual reality system: height, weight, sex, age, body mass, and impairment.

6. The method of claim 3, wherein the trained machine learning model is trained by providing the plurality of stored movement signatures with each of the plurality associated with a stored movement classification.

7. The method of claim 1, wherein determining the movement classification comprises using a data analytics technique to generate data indicative of a classification of the movement signature based on a plurality of stored movement signatures.

8. The method of claim 1, wherein the receiving input from a plurality of sensors comprises receiving position data, acceleration data, and rotational data from each of the plurality of sensors.

9. The method of claim 8, wherein each of the position data, the rotational data, and the acceleration data comprise values for at least three axes.

10. The method of claim 1, wherein the plurality of sensors is a subset of all the sensor positioned on a user of the virtual reality system.

11. A method of providing a therapeutic virtual reality activity, the method comprising:
receiving an input associated with a first movement;
determining a plurality of micromovements based on the first movement;
accessing a plurality of activities, wherein one or more exercise micromovements are associated with each of the plurality of activities;
comparing the plurality of micromovements with the one or more exercise micromovements associated with each of the plurality of activities;
identifying a subset of the plurality of activities based on the comparison of the plurality of micromovements with the one or more exercise micromovements associated with each of the plurality of activities; and
providing the subset of the plurality of activities.

12. The method of claim 11, wherein the first movement is a movement classification determined by:
receiving input from a plurality of sensors for a movement;
generating a first movement signature based on the input from the plurality of sensors for the movement;
determining the using a trained machine learning model to generate data indicative of a classification of the first movement signature based on a plurality of stored movement signatures; and
providing the movement classification.

13. The method of claim 12, wherein the trained machine learning model is trained to receive the movement signature as input and output at least one movement classification describing the movement signature.

14. The method of claim 12, wherein generating the movement signature based on the input from the plurality of sensors comprises charting time against at least one of the following from each of the plurality of sensors: position data, rotation data, and acceleration data.

15. The method of claim 12, wherein the trained machine learning model generates data indicative of the classification of the movement signature further based on at least one of the following criteria associated with a user of the virtual reality system: height, weight, sex, age, body mass, impairment.

16. The method of claim 11, wherein the determining the plurality of micromovements based on the first movement comprises:
receiving input from a plurality of sensors for a movement;
generating a movement signature for the movement based on the input from the plurality of sensors;
identifying one or more breaks in the movement signature; and
extracting the plurality of micromovements from the movement signature based on the identified one or more breaks in the movement signature.

17. The method of claim 16, wherein generating the movement signature based on the input from the plurality of sensors comprises charting time against at least one of the following from each of the plurality of sensors: position data, acceleration data, and rotational data.

18. The method of claim 11, wherein the input associated with the first movement comprises selection of the first movement via user interface.

19. The method of claim 11, wherein the input associated with the first movement comprises input of the first movement via sensors.

20. The method of claim 11, wherein the providing the subset of the plurality of activities comprises:
assigning each of the subset of the plurality of activities a match score based on the comparison of the plurality of micromovements with the one or more exercise micromovements associated with each of the plurality of activities;
ranking the subset of the plurality of activities a match score based on the match score;
selecting an activity of the plurality of activities based on the ranking of the subset of the plurality of activities; and
providing the selected activity of the plurality of activities.

21-40. (canceled)
